(19) United States
(12) Patent Application Publication
(10) Pub. No.: US 2026/0191812 A1
(43) Pub. Date: Jul. 9, 2026

(54) NOVEL ANTI-SENESCENCE COMPOUNDS

(71) Applicant: VITEXIA APS, Copenhagen Ø (DK)

(72) Inventor: Morten WEIDNER, Birkerød (DK)

(73) Assignee: VITEXIA APS, Copenhagen Ø (DK)

(21) Appl. No.: 19/129,837

(22) PCT Filed: Nov. 16, 2023
(86) PCT No.: PCT/EP2023/082132

§ 371 Date: May 14, 2025

(30) Foreign Application Priority Data
Nov. 17, 2022 (EP) 22208070.7

(51) Int. Cl. A61K 31/216; A61K 9/00; A61P 17/00; C07C 69/614; C07C 69/732

(52) U.S. Cl. CPC A61K 31/216; A61K 9/0014; A61P 17/00; C07C 69/614; C07C 69/732

(57) ABSTRACT

The present invention relates to esters of Formula (I) or a pharmaceutically acceptable salt thereof, and their use as a medicament, in particular for the treatment of dermatological diseases or skin cancers. The present invention further relates to use of esters of Formula (I) or a pharmaceutically acceptable salt thereof in cosmetic and nutritive compositions for preventing or reducing signs of aging in healthy subjects.

### FIELD OF THE INVENTION

The present invention relates to novel esters made from mandelic acid (derivatives) and phenylpropanoic acid (derivatives), and their use as a medicament, in particular for the treatment of dermatological diseases. The present invention further relates to cosmetic and nutritive compositions comprising the novel esters for preventing or reducing signs of aging in healthy subjects.

### BACKGROUND OF THE INVENTION

Aging is an inevitable biological process characterized by the progressive deterioration of a variety of physiological functions, rendering the aging person increasingly frail and susceptible to diseases. The aging process is linked to a number of chronic, degenerative, and inflammatory diseases. Aging is characterized by the accumulation of macromolecular damages, impaired tissue renewal, and progressive loss of physiological integrity associated with cellular senescence (Lee et al 2021). Senescence is a state where cells stop proliferating, lose their tissue-specific gene expression and express inflammatory genes, which is termed SASP (senescence-associated secretory phenotype). Senescence is caused by a variety of stresses, accumulation of DNA damage and subsequent epigenetic changes (Orioli et al 2018; Lee et al 2021). The dermis is a connective tissue populated by fibroblasts that are responsible for the synthesis and secretion of matrix components such as collagen and elastin, which maintain the skin architecture and confer elasticity as well as resistance and strength to the tissue (Murphree 2017; Arseni et al 2018). Human dermal fibroblasts are implicated in almost every skin process by interacting with both the epidermal and the other resident dermal cells, such as endothelial, neural, and inflammatory cells as well as adipocytes. Furthermore, the signalling from the dermal compartment is fundamental for the maintenance and homeostasis of epidermal stem cells (Murphree 2017; Sriram et al 2015). Dermal fibroblasts are therefore a primary target cell to counteract skin aging.

The hallmark of skin aging is a shift towards senescent gene expression, which accounts for many of the unwanted structural changes in aging skin resulting in wrinkles, sagging and atrophy (Orioli et al 2018). The age-related shift towards senescence is caused by intrinsic factors, such as telomere shortening, as well as extrinsic factors including UV light, air pollution, cigarette smoke and metabolic stress, which all cause DNA damage. With aging, the dermal fibroblasts acquire a senescent phenotype with significant impact on the skin architecture and function. This phenotype is characterized by the following changes in gene expression:
Lower expression of collagen and elastin genes. Loss of collagen and elastin results in loss of firmness and the formation of deeper wrinkles (Ezure et al 2019).
Higher expression of matrix metallopeptidases (e.g., MMP1 and MMP3). These enzymes degrade the dermal matrix and are selectively overexpressed in relation to traumatic tissue injuries to clear damaged tissue. In aging, their overexpression contributes to the deterioration of the integrity, thickness, and elasticity of the skin (Hornebeck et al., 2003).
Decreased expression of Tissue Inhibitor of Metalloproteinases 1 (TIMP1), which exhibits strong inhibition of matrix metallopeptidases and is released by fibroblasts to control the inflammatory degradation by MMPs. The decreased expression of TIMP1 with fibroblast senescence, both ex vivo and in vivo, contributes to increased dermal connective degradation with aging (Hornebeck et al., 2003).
Higher expression of inflammatory mediator genes. These are characteristic of the senescence associated secretory phenotype (SASP) and besides mediating unspecific inflammation, they play a paracrine role in spreading senescence (Lee et al 2021).
Higher expression of cyclin dependent kinase inhibitor genes, especially p21^{ciP1/waf1} (CDKN1A) in fibroblasts and p16^{INK4a} (CDKN2A) in keratinocytes. Overexpression in senescence causes cell cycle arrest resulting in non-proliferating and inactive cells (Idda et al 2020).
At the cellular level, the shift towards senescence is driven by epigenetic alterations and the accumulation of such changes plays an essential role in the transition into the senescent phenotype (Orioli et al., 2018).
Lower expression of DNA methyltransferase 1 (DNMT1) is characteristic for senescent dermal fibroblasts and keratinocytes. DNMT1 is known as “maintenance DNMT” since it preserves the original methylation patterns during cell divisions. DMNT1 expression inversely correlates with CDKN1A expression and chronological age in human skin samples as well as fibroblasts in culture (Sen et al., 2010).
Lower expression of class III histone deacetylases (HDACs), especially SIRT1, which exerts control of energy metabolism, inflammation, and oxidative stress, as well as mediating cell survival, UV damage response, DNA repair and tissue regeneration (Garcia-Peterson et al 2017). In dermal fibroblasts, SIRT1 expression is significantly reduced with age (Tigges et al 2014; Carlomosti et al., 2017) and, notably, SIRT1 up-regulation or down-regulation results in delayed or accelerated fibroblast senescence, respectively (De Cabo et al., 2015).

While the pharmaceutical industry is increasingly developing new drugs to counteract cellular senescence, the development of true anti-aging anti-senescent skincare products is haltered by the requirement of active cosmetic ingredients to be well-established safe. The ban on animal testing of cosmetic ingredients in the EU and some US states has predominantly limited the chemical scope of new cosmetic ingredients to that of natural chemical entities that are part of the human diet or metabolism. Some dietary compounds like resveratrol and curcumin have been associated with anti-senescent and anti-aging properties, but their effect is only related to a potential delay of senescence and aging. Such compounds have not been shown to reverse senescence and aging or making an aging senescent tissue function as a young. U.S. Ser. No. 10/149,809B2 discloses a skin aging inhibitor comprising resveratrol 3-O-α-glucoside to prevent skin aging such as spots, dullness, wrinkles, sags, and skin roughness. U.S. Pat. No. 8,465,973B2 discloses compositions comprising phosphorylated resveratrol for treating and reducing the symptoms of aging of the skin.

Unlike resveratrol and curcumin, the esters of the present invention have been found not only to delay but to reverse senescence. This makes the esters of the invention suitable in the treatment of a number of inflammatory diseases or disorders, as well as in cometic products to promote skin health and prevent signs of skin aging.

Furthermore, the esters of the present invention were found to possess anti-cancer properties e.g., by enhancing the repair of DNA damage as demonstrated in two models, where skin cancer-related CPDs (cyclobutane pyrimidine dimers) were induced in human skin by ultraviolet (UV) radiation. Skin cancers are cancers that arise from the skin, also known as skin neoplasms. Skin cancer is the most common form of cancer, globally accounting for at least 40% of cancer cases (Cakir et al 2012). There are many types of skin cancers, including basal-cell carcinoma, squamous-cell carcinoma, melanoma, and merkel cell carcinoma among others. The first two, along with a number of less common skin cancers, are known as nonmelanoma skin cancer. Basal-cell carcinoma grows slowly and can damage the tissue around it but is unlikely to spread to distant areas or result in death. It often appears as a painless raised area of skin that may be shiny with small blood vessels running over it or may present as a raised area with an ulcer. Squamous-cell skin cancer is more likely to spread. It usually presents as a hard lump with a scaly top but may also form an ulcer. Melanomas are the most aggressive. Signs include a mole that has changed in size, shape, color, has irregular edges, has more than one color, is itchy or bleeds. The most common type is nonmelanoma skin cancer, which occurs in at least 2-3 million people per year. Of nonmelanoma skin cancers, about 80% are basal-cell carcinomas and 20% squamous-cell carcinomas.

Ultraviolet radiation from sun exposure is believed to be the primary cause of skin cancer. Environmental carcinogens (environmental pollutants) may also cause skin cancer. Examples of environmental carcinogens may include polluted drinking water, poor indoor air quality, chemical pollutants (e.g. asbestos), food chemicals (e.g. dioxins), and ionizing radon radiation. While any region of the body may be affected by skin cancer, it typically occurs on the face, neck, head, and arms. Basal cell and squamous cell carcinoma are responsible for approximately 2700 deaths per year and melanoma is responsible for approximately 7400 deaths per year in the United States (Aggarwal et al 2021).

In case of basal cell or squamous cell carcinoma, several types of options may be given, including surgery, topical chemotherapy, photodynamic therapy, or radiation therapy. In case of melanoma, treatment may include surgery, chemotherapy, isolated limb perfusion, immunotherapy, and radiation therapy. However, some of these treatments are replete with drawbacks such as flu-like symptoms, extreme fatigue, hair-loss, DNA damage, development of secondary cancer, radiation burns in the skin, and cell migration into the bloodstream. Furthermore, no treatment is available for the safe long-term prophylaxis of skin cancer or safe prevention of remission. Thus, there is a need in the art for new effective skin cancer treatments.

### SUMMARY OF THE INVENTION

The present inventor found that the compounds according to the invention possessed anti-senescent properties. More particularly, the compounds of the invention were able to inhibit age-related gene expression (i.e. prevent formation of a senescent cell state) in human fibroblast cells, and even more intriguingly, the compounds were able to revert senescent cells into a non-senescent state. The invention is set forth in the claims.

### DETAILED DESCRIPTION OF THE INVENTION

Unless otherwise defined, all technical and scientific terms used herein have the same meaning as commonly understood by one of ordinary skill in the art to which this invention pertains. The terminology used in the description of the invention herein is for the purpose of describing particular embodiments only and is not intended to be limiting of the invention.

The esters of the present invention are obtained by a formal condensation between the carboxylic acid in 3-phenylpropanoic acid (or a derivative thereof) and the α-hydroxy group in 2-hydroxy-2-phenylacetic acid (or a derivative thereof). In the present context, “2-hydroxy-2-phenylacetic acid” (IUPAC name) may be referred to simply as “mandelic acid”. Likewise, “3-phenylpropanoic acid” (IUPAC name) may be referred to simply as “phenylpropanoic acid”. The prefix “(R)” or “(S)” has the usual meaning in the art and indicates the (R)-enantiomer or the (S)-enantiomer, respectively. Likewise, the prefix “(RS)” refers to a racemic mixture.

In the present context, a “non-therapeutic benefit” refers to the effect of an ester in a cosmetic or nutritive composition of the invention to maintain, reduce or enhance physiological processes or parameters within the normal physiological range in a healthy subject. Non-limiting examples of non-therapeutic benefits are inhibition or reversal of cellular senescence, inhibition or reversal of aging, reduction of facial wrinkles, maintenance or improvement of skin, maintenance or improvement of muscle endurance or muscle strength, optimizing sports endurance, maintenance or improvement of mitochondrial function, counteraction of aging or signs of aging.

In the present context, the term “treatment” should be understood in the broadest sense as prevention, amelioration, or treatment of a disease or disorder. Thus, treatment is also intended to include prophylactic treatment of a disease or disorder. Thus, “treatment” refers to a therapeutic effect (i.e. a medical or therapeutic benefit) in a disease or disorder resulting in a reduction, alleviation, mitigation or decrease of at least one clinical symptom associated with the disease or disorder, or a delay in the progression of the disease or disorder, and/or prevention or delay of onset of a disease or disorder. Hence, treatment should be understood as the effect of a medicament on pathological processes with the purpose of preventing or counteracting a disease or disorder, or a physiological process potentially leading to a disease or disorder. Non-limiting examples of therapeutic effects are inhibition or reversal of disease-related cellular senescence, improvement of mitochondrial biogenesis and function, reduction or prevention of inflammation, alleviation of pain, improvement of wound healing, inhibition or reversal of disease-related cellular malignancy, inhibition of malignant cell growth and cytotoxicity towards malignant cells.

The term “an effective amount” refers to an amount of an ester of the present invention that is sufficient to produce the desired effect. The effective amount will vary with the application for which compositions are being employed, the age and physical condition of the subject, the severity of the disease or disorder, the duration of the treatment, the nature of any concurrent treatment, the carrier used, and similar factors within the knowledge and expertise of those skilled in the art.

The term “support” is used interchangeably with the terms “maintain”, “restore” or “preserve”. The term “decrease” is used interchangeably with the terms “lower”, “counteract”, “decrease” or “reduce”. The term “normalize” is used interchangeably with the terms “regulate” or “modulate”. The term “improve” is used interchangeably with the terms “enhance”, “promote”, “stimulate”, “increase” or “raise”.

In the present context, it should be understood that the esters of the invention may be in the form of pharmaceutically acceptable salts. Suitable examples may be found e.g., in Remington's Pharmaceutical Sciences, 17^{th} edition. Likewise, various solvates of the esters or a pharmaceutically acceptable salt thereof are also within the scope of the invention.

In the present context, it should be understood that alkyl and alkoxy groups may be linear, branched, or cyclic. As a non-limiting example, a C₃-alkyl may be a linear n-propyl (—CH₂CH₂CH₃), an isopropyl (—CH(CH₃)₂), or a cyclopropyl. Most preferably, alkyl and/or alkoxy groups are linear or branched.

The present invention will now be described in more detail. It should be appreciated that the invention can be embodied in different forms and should not be construed as limited to the embodiments set forth herein.

The present inventor found that the compounds of Formula (I) possessed anti-senescent properties. More particularly, the compounds of the invention were able to inhibit age-related gene expression (i.e. prevent formation of a senescent cell state) in human fibroblast cells, and even more intriguingly, the compounds were able to revert senescent cells into a non-senescent state. These observations make the compounds suitable in the treatment of a range of diseases and disorders, in particular in the treatment of dermatological diseases, rheumatological diseases, and wounds. Furthermore, the findings make the compounds suitable in cosmetic and nutritional compositions for preventing signs and symptoms of aging in healthy subjects.

In a first aspect, the invention relates to a compound of the general Formula (I) or a pharmaceutical acceptable salt thereof

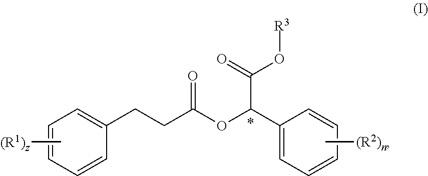

wherein
* denotes the (S) or (R) enantiomer or any mixture thereof;
z is an integer of 0, 1, 2, 3, 4 or 5, and R¹ is/are independently selected from F, OH, and C₁-C₆ alkoxy;
n is an integer of 0, 1, 2, 3, 4 or 5, and R² is/are independently selected from F, OH, and C₁-C₆ alkoxy;
R³ is selected from H and C₁-C₈ alkyl.

### Number of (R¹) Groups (z)

The inventor found that the compounds according to the invention may contain up to 5 (i.e. z=5) (R¹) substituents. In an embodiment, z is an integer of 0-4, preferably z is an integer of 0-3, more preferably z is an integer of 0-2, most preferably z is an integer of 1-3. Thus, it is most preferred that the phenylpropanoic acid part of the ester has one or two substituents.

### Number of (R²) Groups (n)

The inventor found that the compounds according to the invention may contain up to 5 (i.e. n=5) (R²) substituents. In an embodiment, n is an integer of 0-4, preferably n is an integer of 0-3, more preferably n is an integer of 0-2, most preferably n is an integer of 0-1. Thus, it is most preferred that the mandelic acid part of the ester is unsubstituted or has one substituent (R²).

Most preferably, the total number of R¹ and R² groups (i.e. n+z) does not exceed 6.

### Type of Substituent (R¹)

The inventor found that the aromatic ring in the phenylpropanoic acid part of the ester tolerated fluorine (—F), hydroxy (—OH) substituents and/or alkoxy substituents (—O-alkyl). It should be appreciated that these substituents can also be replaced with bioisosters known in the art. Thus, in an embodiment, R¹ (if present) is/are independently selected from F, OH, and C₁-C₆ alkoxy. In an embodiment, R¹ (if present) is/are independently selected from OH, and C₁-C₅ alkoxy. In a preferred embodiment, R¹ (if present) is/are independently selected from OH, and C₁-C₄ alkoxy. In another preferred embodiment, R¹ (if present) is/are independently selected from OH, and C₁-C₃ alkoxy. In another preferred embodiment, R¹ (if present) is/are independently selected from OH, and C₁-C₂ alkoxy. In yet another preferred embodiment, R¹ (if present) is/are independently selected from OH, and methoxy (OMe). In the most preferred embodiment, R¹ (if present) is/are independently selected from OH, and C₁-C₃ alkoxy.

### Type of Substituent (R^{Z})

The inventor found that the aromatic ring in the mandelic acid part tolerated fluorine (—F), hydroxy (—OH) substituents, and/or alkoxy substituents (—O-alkyl). It should be appreciated that these substituents can also be replaced with bioisosters known in the art. Thus, in an embodiment, R² (if present) is/are independently selected from F, OH, and C₁-C₆ alkoxy. In an embodiment, R² (if present) is/are independently selected from OH, and C₁-C₅ alkoxy. In a preferred embodiment, R² (if present) is/are independently selected from OH, and C₁-C₄ alkoxy. In another preferred embodiment, R² (if present) is/are independently selected from OH, and C₁-C₃ alkoxy. In another preferred embodiment, R² (if present) is/are independently selected from OH, and C₁-C₂ alkoxy. In yet another preferred embodiment, R² (if present) is/are independently selected from OH, and methoxy (OMe). In the most preferred embodiment, R² (if present) is/are independently selected from OH, and C₁-C₃ alkoxy.

### Position of the R¹ Substituent

In a preferred embodiment, (R¹), when present, is/are located in position 3, 4 and/or 5 as shown in Formula (I) below (i.e. meta and/or para).

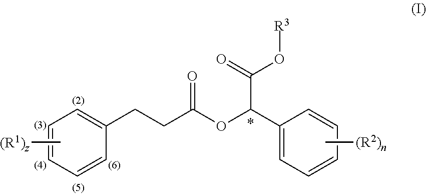

In another preferred embodiment, (R¹), when present, is/are located in position 2 and/or 6 as shown in Formula (I) below (i.e. ortho).

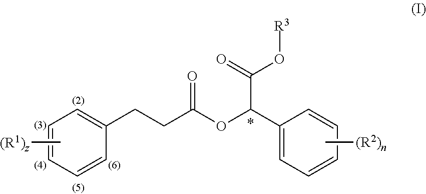

### Position of the R² Substituent

In a preferred embodiment, R² group(s), when present, is/are located in position 3, 4 and/or 5 as shown in Formula (I) below (i.e. meta and/or para). Most preferably R² is located in position 4.

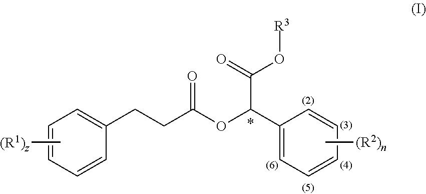

### R³ Substituent

In an embodiment, R³ is selected as H or C₁-C₇ alkyl, more preferably, R³ is selected as H or C₁-C₆ alkyl. In a preferred embodiment, R³ is selected as H or C₁-C₅ alkyl. In a more preferred embodiment, R³ is selected as H or C₁-C₄ alkyl. In a highly preferred embodiment, R³ is selected as H or C₁-C₃ alkyl. In a more highly preferred embodiment, R³ is selected as H or C₁-C₂ alkyl. In an even more highly preferred embodiment, R³ is selected as H or C₁ alkyl (methyl). In a most preferred embodiment, R³ is selected as H.

### Preferred Embodiments

In an embodiment, z is an integer of 0, 1, 2, or 3; R¹ (if present) is/are independently selected from OH, and C₁-C₃ alkoxy; n is an integer of 0, 1, 2, or 3; R² (if present) is/are independently selected from OH and C₁-C₃ alkoxy; R³ is selected as H or C₁-C₃ alkyl.

In another embodiment, z is an integer of 0, 1, 2, or 3; R¹ (if present) is/are independently selected from OH, and C₁-C₃ alkoxy; n is an integer of 0, 1, 2, or 3; R² (if present) is/are independently selected from OH and C₁-C₂ alkoxy; R³ is selected as H or C₁-C₃ alkyl.

In another embodiment, z is an integer of 0, 1, 2, or 3; R¹ (if present) is/are independently selected from OH, and C₁-C₃ alkoxy; n is an integer of 0, 1, 2, or 3; R² (if present) is/are independently selected from OH and methoxy (OMe); R³ is selected as H or C₁-C₃ alkyl.

In another embodiment, z is an integer of 0, 1, 2, or 3; R¹ (if present) is/are independently selected from OH, and C₁-C₂ alkoxy; n is an integer of 0, 1, 2, or 3; R² (if present) is/are independently selected from OH and C₁-C₃ alkoxy; R³ is selected as H or C₁-C₃ alkyl.

In another embodiment, z is an integer of 0, 1, 2, or 3; R¹ (if present) is/are independently selected from OH, and methoxy (OMe); n is an integer of 0, 1, 2, or 3; R² (if present) is/are independently selected from OH and C₁-C₃ alkoxy; R³ is selected as H or C₁-C₃ alkyl.

In an embodiment, z is an integer of 0, 1, or 2; R¹ (if present) is/are independently selected from OH, and C₁-C₃ alkoxy; n is an integer of 0, 1, or 2; R² (if present) is/are independently selected from OH and C₁-C₃ alkoxy; R³ is selected as H or C₁-C₃ alkyl.

In another embodiment, z is an integer of 0, 1, or 2; R¹ (if present) is/are independently selected from OH, and C₁-C₃ alkoxy; n is an integer of 0, 1, or 2; R² (if present) is/are independently selected from OH and C₁-C₂ alkoxy; R³ is selected as H or C₁-C₃ alkyl.

In another embodiment, z is an integer of 0, 1, or 2; R¹ (if present) is/are independently selected from OH, and C₁-C₃ alkoxy; n is an integer of 0, 1, or 2; R² (if present) is/are independently selected from OH and methoxy (OMe); R³ is selected as H or C₁-C₃ alkyl.

In another embodiment, z is an integer of 0, 1, or 2; R¹ (if present) is/are independently selected from OH, and C₁-C₂ alkoxy; n is an integer of 0, 1, or 2; R² (if present) is/are independently selected from OH and C₁-C₃ alkoxy; R³ is selected as H or C₁-C₃ alkyl.

In another embodiment, z is an integer of 0, 1, or 2; R¹ (if present) is/are independently selected from OH, and methoxy (OMe); n is an integer of 0, 1, or 2; R² (if present) is/are independently selected from OH and C₁-C₃ alkoxy; R³ is selected as H or C₁-C₃ alkyl.

In another embodiment, z is an integer of 0, 1, or 2; R¹ (if present) is/are independently selected from OH, and methoxy (OMe); n is an integer of 0, 1, or 2; R² (if present) is/are independently selected from OH and methoxy (OMe); R³ is selected as H or C₁-C₃ alkyl.

In a highly preferred embodiment, z is an integer of 1, 2, or 3; R¹ (if present) is/are independently selected from OH and C₁-C₃ alkoxy; n is an integer of 0, 1, or 2; R² (if present) is/are independently selected from OH and C₁-C₃ alkoxy; R³ is selected as H or C₁-C₃ alkyl.

In a highly preferred embodiment, z is an integer of 0, 1, 2, 3, 4 or 5 and R¹ is/are independently selected from F, OH, and C₁-C₃ alkoxy; n is an integer of 0, 1, 2, 3, 4 or 5 and R² is/are independently selected from F, OH, and C₁-C₃ alkoxy; R³ is selected from H and C₁-C₈ alkyl; with the proviso that at least one of R¹ or R² is OH.

In another highly preferred embodiment, z is an integer of 1, 2, or 3; R¹ (if present) is/are independently selected from OH and C₁-C₃ alkoxy; n is an integer of 0, 1, or 2; R² (if present) is/are independently selected from OH and C₁-C₃ alkoxy, and wherein R² is present in position 3, 4 and/or 5 (i.e. meta or para); R³ is selected as H or C₁-C₃ alkyl.

In another highly preferred embodiment, z is an integer of 1, 2, or 3; R¹ (if present) is/are independently selected from OH and C₁-C₃ alkoxy; n is an integer of 0, 1, or 2; R² (if present) is/are independently selected from OH and C₁-C₃ alkoxy, and wherein R² is present in position 2, and/or 6 (i.e. ortho); R³ is selected as H or C₁-C₃ alkyl.

In a highly preferred embodiment, the compound of Formula (I) is selected from a list consisting of

mandelic acid 2-hydroxy-phenylpropanoate, mandelic acid 3-hydroxy-phenylpropanoate, mandelic acid 4-hydroxy-phenylpropanoate, mandelic acid 3,4-dihydroxyphenylpropanoate, mandelic acid 3,4,5-trihydroxyphenylpropanoate, mandelic acid 2-methoxyphenylpropanoate, mandelic acid 3-methoxyphenylpropanoate, mandelic acid 4-methoxyphenylpropanoate, mandelic acid 4-hydroxy-3-methoxyphenylpropanoate, mandelic acid 4-hydroxy-3,5-dimethoxyphenyl-propanoate, mandelic acid 2,3-dimethoxyphenylpropanoate, mandelic acid 2,4-dimethoxyphenylpropanoate, mandelic acid 2,5-dimethoxyphenylpropanoate, mandelic acid 3,4-dimethoxyphenylpropanoate, mandelic acid 3,5-dimethoxyphenylpropanoate, mandelic acid 2,3,4-trimethoxyphenylpropanoate, mandelic acid 3,4,5-trimethoxyphenylpropanoate, mandelic acid 4-ethoxyphenylpropanoate, mandelic acid 4-propoxyphenylpropanoate, mandelic acid 2-hydroxy-phenylpropanoate, mandelic acid 3-hydroxy-phenylpropanoate, mandelic acid 4-hydroxy-phenylpropanoate, mandelic acid 3,4-dihydroxyphenylpropanoate, mandelic acid 2-methoxyphenylpropanoate, mandelic acid 3-methoxyphenylpropanoate, mandelic acid 4-methoxyphenylpropanoate, mandelic acid 4-hydroxy-3-methoxyphenylpropanoate, mandelic acid 3,5-dimethoxyphenylpropanoate, mandelic acid 3,4,5-trimethoxyphenylpropanoate, mandelic acid 4-ethoxy-phenylpropanoate, mandelic acid 4-propoxy-phenylpropanoate, 4-hydroxymandelic acid 2-hydroxyphenylpropanoate, 4-hydroxymandelic acid 3-hydroxyphenylpropanoate, 4-hydroxymandelic acid 4-hydroxyphenylpropanoate, 4-hydroxymandelic acid 3,4-dihydroxyphenyl-propanoate, 4-hydroxymandelic acid 2-methoxyphenylpropanoate, 4-hydroxymandelic acid 3-methoxyphenylpropanoate, 4-hydroxymandelic acid 4-methoxyphenylpropanoate, 4-hydroxymandelic acid 4-hydroxy-3-methoxyphenyl-propanoate, 4-hydroxymandelic acid 3,5-dimethoxyphenyl-propanoate, 4-hydroxymandelic acid 4-ethoxyphenylpropanoate, 4-hydroxymandelic acid 4-propoxyphenylpropanoate, 4-methoxymandelic acid 2-hydroxy-phenylpropanoate, 4-methoxymandelic acid 3-hydroxy-phenylpropanoate, 4-methoxymandelic acid 4-hydroxyphenyl-propanoate, 4-methoxymandelic acid 3,4-dihydroxyphenylpropanoate, 4-methoxymandelic acid 2-methoxyphenylpropanoate, 4-methoxymandelic acid 3-methoxyphenylpropanoate, 4-methoxymandelic acid 4-methoxyphenylpropanoate, 4-methoxymandelic acid 4-hydroxy-3-methoxyphenyl-propanoate, 4-methoxymandelic acid 3,5-dimethoxyphenyl-propanoate, 4-methoxymandelic acid 4-ethoxyphenylpropanoate, 4-methoxymandelic acid 4-propoxyphenylpropanoate, 4-propoxymandelic acid 2-hydroxyphenylpropanoate, 4-propoxymandelic acid 3-hydroxyphenylpropanoate, 4-propoxymandelic acid 4-hydroxyphenyl-propanoate, 4-propoxymandelic acid 3,4-dihydroxyphenylpropanoate, 4-propoxymandelic acid 2-methoxyphenylpropanoate, 4-propoxymandelic acid 3-methoxyphenylpropanoate, 4-propoxymandelic acid 4-methoxyphenylpropanoate, 4-propoxymandelic acid 4-hydroxy-3-methoxyphenyl-propanoate, 4-propoxymandelic acid 3,5-dimethoxyphenyl-propanoate, 4-propoxymandelic acid 4-ethoxyphenylpropanoate, 4-propoxymandelic acid 4-propoxyphenylpropanoate, 4-hydroxy-3-methoxymandelic acid 2-hydroxyphenylpropanoate, 4-hydroxy-3-methoxy-mandelic acid 3-hydroxy-phenylpropanoate, 4-hydroxy-3-methoxy-mandelic acid 4-hydroxy-phenylpropanoate, 4-hydroxy-3-methoxy-mandelic acid 3,4-dihydroxy-phenylpropanoate, 4-hydroxy-3-methoxy-mandelic acid 2-methoxy-phenylpropanoate, 4-hydroxy-3-methoxy-mandelic acid 3-methoxy-phenylpropanoate, 4-hydroxy-3-methoxy-mandelic acid 4-methoxy-phenylpropanoate, 4-hydroxy-3-methoxy-mandelic acid 4-hydroxy-3-methoxy-phenylpropanoate, 4-hydroxy-3-methoxy-mandelic acid 3,5-dimethoxy-phenylpropanoate, 4-hydroxy-3-methoxy-mandelic acid 4-ethoxy-phenylpropanoate, and 4-hydroxy-3-methoxy-mandelic acid 4-propoxy-phenylpropanoate, methyl mandelate 4-hydroxy-3-methoxy-phenylpropanoate, methyl mandelate 4-hydroxy-3-methoxy-phenylpropanoate, methyl mandelate 4-hydroxy-3-methoxy-phenylpropanoate, ethyl mandelate 4-hydroxy-3-methoxy-phenylpropanoate, ethyl mandelate 4-hydroxy-3-methoxy-phenylpropanoate, isopropyl mandelate 4-hydroxy-3-methoxy-phenylpropanoate, isoamyl mandelate 4-hydroxy-3-methoxy-phenylpropanoate, benzyl mandelate 3,4-dihydroxyphenylpropanoate, phenethyl mandelate 3,4-dihydroxyphenylpropanoate, wherein the compounds may be (S) or (R) enantiomer or any mixture thereof.

In a more highly preferred embodiment the compound is mandelic acid 3-hydroxyphenylpropanoate, mandelic acid 4-hydroxyphenylpropanoate, mandelic acid 3,4-dihydroxyphenylpropanoate mandelic, mandelic acid 4-hydroxy-3-methoxyphenylpropanoate, acid 2-methoxyphenylpropanoate, mandelic acid 3-methoxyphenylpropanoate, mandelic acid 4-methoxyphenylpropanoate, mandelic acid 4-ethoxyphenylpropanoate, mandelic acid 4-propoxyphenylpropanoate, mandelic acid 2,5-dimethoxyphenylpropanoate, mandelic acid 3,5-dimethoxyphenylpropanoate, mandelic acid 3,4,5-trimethoxyphenylpropanoate, 4-hydroxymandelic acid 2-methoxyphenylpropanoate, 4-hydroxymandelic acid 3-hydroxyphenylpropanoate, 4-hydroxymandelic acid 4-hydroxyphenylpropanoate, 4-hydroxymandelic acid 4-hydroxy-3-methoxyphenylpropanoate, 4-hydroxymandelic acid 3,5-dimethoxy-phenylpropanoate, 4-methoxymandelic acid 2-methoxyphenylpropanoate, 4-methoxymandelic acid 4-methoxyphenylpropanoate, 4-methoxymandelic acid 3,5-dimethoxyphenylpropanoate, 4-methoxymandelic acid 4-ethoxyphenylpropanoate, 4-propoxymandelic acid 4-hydroxyphenylpropanoate, 4-propoxymandelic acid 2-methoxyphenylpropanoate, 4-propoxymandelic acid 3-methoxyphenylpropanoate, 4-propoxymandelic acid 3,5-dimethoxy-phenylpropanoate, 4-propoxymandelic acid 4-hydroxy-3-methoxyphenylpropanoate, 4-propoxymandelic acid 4-ethoxyphenylpropanoate, 4-propoxymandelic acid 4-propoxyphenylpropanoate, 4-hydroxy-3-methoxymandelic acid 4-methoxyphenylpropanoate, 4-hydroxy-3-methoxymandelic acid 3,5-dimethoxyphenylpropanoate and 4-hydroxy-3-methoxy-mandelic acid 4-ethoxyphenylpropanoate, wherein the compounds may be (S) or (R) enantiomer or any mixture thereof.

In a most preferred embodiment the compound is 2-((3-(4-hydroxyphenyl)propanoyl)oxy)-2-phenylacetic acid (i.e. mandelic acid 4-hydroxyphenylpropanoate), 2-((3-(3,4-dihydroxyphenyl)propanoyl)oxy)-2-phenylacetic acid (i.e. mandelic acid 3,4-dihydroxyphenyl-propanoate) or 2-((3-(4-hydroxy-3-methoxyphenyl)propanoyl)oxy)-2-phenylacetic acid (i.e. mandelic acid 4-hydroxy-3-methoxyphenylpropanoate).

### Medical Use

The compounds of the present invention may be used for the treatment of a disease or disorder associated with increased levels of cellular senescence, in particular dermatological diseases, rheumatological diseases, and/or wound healing. The compounds of the present invention may also be used for the treatment of cancers, in particular skin cancers.

Thus, in a second aspect, the present invention relates to a compound of Formula (I) or a pharmaceutically acceptable salt thereof for use as a medicament. In an embodiment, the present invention relates to a compound of Formula (I) or a pharmaceutically acceptable salt thereof for use as a medicament in the treatment of a dermatological disease, a rheumatological disease, a skin cancer and/or a wound.

Hence, the present invention also relates to a method of treating a dermatological disease, a rheumatological disease, a skin cancer and/or a wound, the method comprising administering a therapeutic effective amount of a compound of Formula (I) or a pharmaceutically acceptable salt thereof.

In an embodiment, the present invention relates to a compound of Formula (I) or a pharmaceutically acceptable salt thereof for use in the treatment of a dermatological disease or disorder selected from the group consisting of asteatotic eczema, stasis dermatitis, lichen simplex chronicus, seborrheic dermatitis, seborrhea, psoriasis, atopic dermatitis, infantile eczema, childhood eczema, adult eczema, keratosis pilaris, ichthyosis vulgaris, hand and foot dermatitis, keratoconus, pompholyx, discoid eczema, nummular eczema, allergic contact dermatitis, irritant contact dermatitis, overtreatment dermatitis, hand eczema, and sun damage.

In an embodiment, the present invention relates to a compound of Formula (I) or a pharmaceutically acceptable salt thereof for use in the treatment of a rheumatological disease or disorder selected from the group consisting of rheumatoid arthritis, osteoarthritis, ankylosing spondylitis, Reiter's syndrome, psoriatic arthritis, gout, juvenile chronic arthritis, enteropathic synovitis, infective arthritis, soft tissue rheumatism and fibromyalgia.

In an embodiment, the present invention relates to a compound of Formula (I) or a pharmaceutically acceptable salt thereof for use in the treatment of a wound selected from an acute or chronic dermal wound; an acute or chronic wound related to body tissue selected among muscles, fat, bones, inner organs, nerve tissue, cartilage, joints, arteries, veins, the gastro-intestinal tract, mucus membranes and eyes; an acute wound selected among traumatic wounds, surgical wounds, infected wounds, mucus membranes wounds, burn wounds, wounds caused by an underlying condition and corneal ulcers; a chronic wound selected among surgical wounds, traumatic wounds, burn wounds, infected or contaminated wounds, venous ulcers, arterial ulcers, mixed venous-arterial ulcers, pressure ulcers, diabetic ulcers, neuropathic ulcers, fistulas, immunological ulcers, malignant ulcers, dermatitis ulcers, radiation ulcers, pyoderma gangrenosum and skin graft treated wounds; a traumatic wound selected among cuts, crushes, punctures, lacerations, contusions, abrasions and avulsions; a wound which is poorly and/or slowly healing.

In an embodiment, the present invention relates to a compound of Formula (I) or a pharmaceutically acceptable salt thereof for use in the treatment of a skin cancer selected from the group consisting of basal cell carcinoma, squamous cell carcinoma, melanoma, merkel cell carcinoma, cutaneous T-cell lymphoma, dermatofibrosarcoma protuberans, merkel cell carcinoma and sebaceous carcinoma.

In an embodiment, the present invention relates to a compound of Formula (I) or a pharmaceutically acceptable salt thereof for use in the treatment of a pre-cancerous state, e.g. actinic keratoses.

### Pharmaceutical Composition

The compounds of the present invention may be in the form of a pharmaceutical composition. The pharmaceutical composition may comprise a pharmaceutically acceptable carrier and/or one or more excipients commonly used in the art. The pharmaceutical composition may be formulated as e.g. emulsions, liniments, solutions, gels, foams, tablets, capsules, powders, etc.

Cosmetic or Nutritive Compositions and their Use

The compounds of the present invention may also be used in cosmetic or nutritive compositions for preventing or reducing signs of aging in healthy subjects. The cosmetic or nutritive composition may be used for reducing or counteracting wrinkles/fine lines, age spots, hyperpigmentation, and/or solar lentigines. The cosmetic or nutritive composition may also be used for increasing the smoothness of the skin, the density of the dermis, the cutaneous microcirculation, and/or skin hydration.

Thus, in a third aspect the present invention relates to the use of a compound of Formula (I) or a pharmaceutically acceptable salt thereof in reducing or counteracting wrinkles/fine lines, age spots, hyperpigmentation, and/or solar lentigines, or in increasing the smoothness of the skin, the density of the dermis, the cutaneous microcirculation, and/or skin hydration. Hence, the present invention also relates to the method for reducing or counteracting wrinkles/fine lines, age spots, hyperpigmentation, and/or solar lentigines, or increasing the smoothness of the skin, the density of the dermis, the cutaneous microcirculation, and/or skin hydration, the method comprising administering an effective amount of a compound of Formula (I) or a pharmaceutically acceptable salt thereof to a subject. The compound may be administered by e.g. applying a cosmetic composition to at least part of the skin or by intake/consumption of a nutritive composition.

In the present context, the term “cosmetic composition” refers to a cosmetic or skincare product that has an ester of the invention added to provide a non-therapeutic benefit in healthy subjects. Non-limiting examples include products commonly referred to as “dermocosmetics” or “anti-aging skincare”, e.g. designed to counteract the signs of aging or the aging process itself in healthy subjects. The regulatory definition and denomination of such products may vary in different parts of the world and are under regular change. Non-limiting examples of such products include creams, lotions, gels, serums, liniments, foams, pastes, sprays, serums, solutions, powders, shampoos, and conditioners. In the present context, the term “nutritive composition” refers to a food or non-food product that has an ester of the invention added to provide a non-therapeutic physiological benefit. Non-limiting examples include products commonly referred to as functional foods, food supplements, dietary supplements, nutritional supplements, nutraceuticals, or medical foods. The regulatory definition and denomination of such products vary significantly in different parts of the world and is under regular change. A nutritive composition in the form of a “food product” may be in the form of specialized food preparations or common foods or beverages, including soft drinks, juices, smoothies, dairy products, etc. A nutritive product in the form of a “non-food product” may be in the form of e.g. tablets, capsules, powders, chewing gum and lozenges.

### EXPERIMENTAL SECTION

### Example 1

### Screening

(RS)-Mandelic acid 3,4-dihydroxyphenylpropanoate was identified as a modulator for the pharmacological targets Histone acetyl transferase p300 (p300), Phosphodiesterase 4 (PDE4) and Kelch-like ECH-associated Protein 1 (KEAP1)/Nuclear factor erythroid 2-related factor 2 (NRF2). Analogs of (RS)-Mandelic acid 3,4-dihydroxyphenylpropanoate (i.e. compounds of Formula (I)) were identified by applying artificial intelligence in the screening of thousands of analogs for possible effects on the molecular targets. Analysis was performed with a combination of in silico tools including Schrödinger's Small Molecule Suite (shape screening, pharmacophore screening and docking analysis) as well as ADMET Predictor from Simulations Plus.

Exemplary compounds of Formula (I) were subsequently synthesized and tested for their ability to modulate such targets (see Example 2 for data) and counteract the senescent phenotype in three models of dermal aging reflecting intrinsic as well as extrinsic induction of senescence (see Example 3 for data). Furthermore, the ability to enhance DNA repair was demonstrated in two models of UV-induced cyclobutane pyrimidine dimer DNA lesions with therapeutic relevance for senescence as well as skin cancer.

### Materials and Chemicals

All chemicals employed were of standard analytical or synthesis grade. ((R)-mandelic acid (prod. no. 154210), (S)-mandelic acid (prod. no. 778052), isoamyl (RS)-mandelate (prod. no. S351512), phenethyl (RS)-mandelate (prod. no. S679380), benzyl (RS)-mandelate (prod. no. S679399) and Oxalyl chloride (prod. no. 221015) were purchased from Merck, Germany. (RS)-4-hydroxymandelic acid (prod. no. TCIAH0660) was purchased from Avantor, Denmark. (RS)-4-methoxymandelic acid (prod. no. BD98560), (RS)-4-propoxymandelic acid (prod. no. BD22647), (RS)-4-Hydroxy-3-methoxymandelic acid (prod. no. BD6743), methyl (R)-mandelate (prod. no. BD6677), methyl (S)-mandelate (prod. no. BD32621), ethyl (R)-mandelate (prod. no. BD127771), ethyl (S)-mandelate (prod. no. BD135652) were purchased from BLD Pharmatech, Germany. All hydroxyphenylpropanoic acids were provided by Zentexia ApS, Denmark. Solid phase extraction was carried out with Discovery® DSC-18 SPE Tubes purchased from Merck, Denmark (prod. no. 52609-U).

### Synthesis of the Compounds (Esters) of the Invention

Exemplary esters of the invention were prepared according to the following procedure: 0.4 mmol of the phenylpropanoic acid (derivative) was dissolved in a mixture of methylene chloride and tetrahydrofuran. 0.45 mmol Oxalyl chloride was added under argon, and the mixture was allowed to react for 5-10 minutes at ambient temperature yielding the acid chloride. 0.4 mmol of the mandelic acid (derivative) was dissolved in tetrahydrofuran and added to the acid chloride solution under argon, and the mixture was allowed to react for 45 minutes at ambient temperature yielding the ester of the invention. The ester solution was dried on a rotary evaporator and dissolved in a mixture of ethyl acetate and methylene chloride. The solution was transferred to a separatory funnel and washed four times with brine. The organic phase was dried with anhydrous sodium sulfate and dried on a rotary evaporator to yield the ester of the invention. Optionally, a further purification was carried out by employing a standard solid phase extraction procedure, where a solution of the crude ester was loaded on a Discovery® DSC-18 SPE Tube and eluted with gradually increasing ethanol from 100% water to 99.5% ethanol. The purity of the ester of the invention is evaluated with HPLC and LCMS employing an Agilent 1200 HPLC equipped with a DAD detector and a 4610 QQQ detector (ESI negative mode). Elution was carried out with a gradient of water and acetonitrile (both containing 0.1% formic acid) on a Poroshell 120 SB-C18 column (3×150 mm, 2.7 μm) from Agilent Technologies.

The following esters of the invention were prepared:

| | | Primary ion |
|---|---|---|
| | | in LCMS [M − |
| Ester of the invention | Structure | H]− |
| (R)-mandelic acid phenylpropanoate | 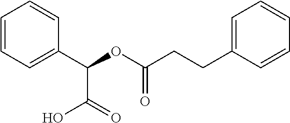 [SMILES: O=C(CCc1ccccc1)O[C@@H](C(=O)O)c1ccccc1] | 283.1 |
| (R)-mandelic acid 2-hydroxy- phenylpropanoate | 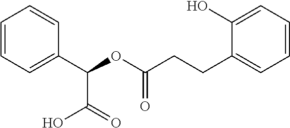 [SMILES: O=C(CCc1ccccc1O)O[C@@H](C(=O)O)c1ccccc1] | 299.1 |
| (R)-mandelic acid 3-hydroxy- phenylpropanoate | 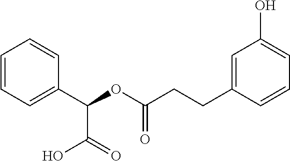 [SMILES: O=C(CCc1cccc(O)c1)O[C@@H](C(=O)O)c1ccccc1] | 299.1 |
| (R)-mandelic acid 4-hydroxy- phenylpropanoate | 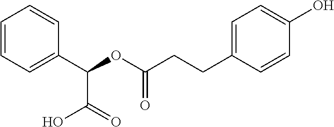 [SMILES: O=C(CCc1ccc(O)cc1)O[C@@H](C(=O)O)c1ccccc1] | 299.1 |
| (R)-mandelic acid 3,4- dihydroxyphenylpropanoate | 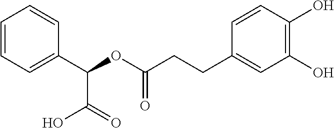 [SMILES: O=C(CCc1ccc(O)c(O)c1)O[C@@H](C(=O)O)c1ccccc1] | 315.1 |
| (R)-mandelic acid 3,4,5- trihydroxyphenylpropanoate | 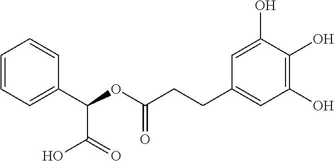 [SMILES: O=C(CCc1cc(O)c(O)c(O)c1)O[C@@H](C(=O)O)c1ccccc1] | 331.1 |
| (R)-mandelic acid 2- methoxyphenylpropanoate | 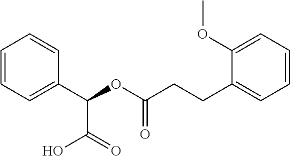 [SMILES: COc1ccccc1CCC(=O)O[C@@H](C(=O)O)c1ccccc1] | 313.1 |
| (R)-mandelic acid 3- methoxyphenylpropanoate | 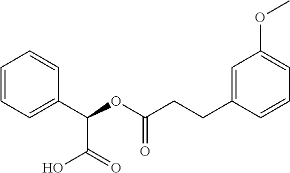 [SMILES: COc1cccc(CCC(=O)O[C@@H](C(=O)O)c2ccccc2)c1] | 313.1 |
| (R)-mandelic acid 4- methoxyphenylpropanoate | 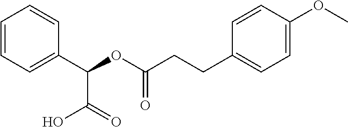 [SMILES: COc1ccc(CCC(=O)O[C@@H](C(=O)O)c2ccccc2)cc1] | 313.,1 |
| (R)-mandelic acid 4-hydroxy-3- methoxyphenylpropanoate | 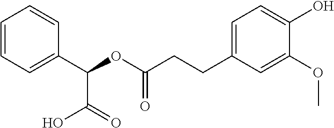 [SMILES: COc1cc(CCC(=O)O[C@@H](C(=O)O)c2ccccc2)ccc1O] | 329.1 |
| (R)-mandelic acid 4-hydroxy-3,5- dimethoxyphenyl-propanoate | 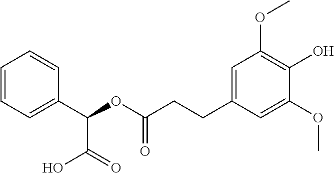 [SMILES: COc1cc(CCC(=O)O[C@@H](C(=O)O)c2ccccc2)cc(OC)c1O] | 359.1 |
| (R)-mandelic acid 2,3- dimethoxyphenylpropanoate | 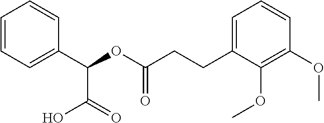 [SMILES: COc1cccc(CCC(=O)O[C@@H](C(=O)O)c2ccccc2)c1OC] | 343.1 |
| (R)-mandelic acid 2,4- dimethoxyphenylpropanoate | 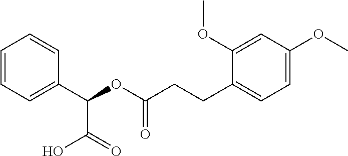 [SMILES: COc1ccc(CCC(=O)O[C@@H](C(=O)O)c2ccccc2)c(OC)c1] | 343.1 |
| (R)-mandelic acid 2,5- dimethoxyphenylpropanoate | 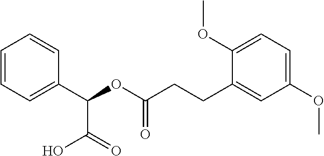 [SMILES: COc1ccc(OC)c(CCC(=O)O[C@@H](C(=O)O)c2ccccc2)c1] | 343.1 |
| (R)-mandelic acid 3,4- dimethoxyphenylpropanoate | 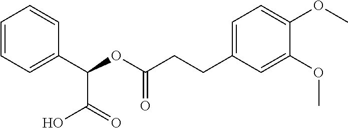 [SMILES: COc1ccc(CCC(=O)O[C@@H](C(=O)O)c2ccccc2)cc1OC] | 343.1 |
| (R)-mandelic acid 3,5- dimethoxyphenylpropanoate | 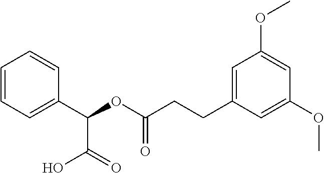 [SMILES: COc1cc(CCC(=O)O[C@@H](C(=O)O)c2ccccc2)cc(OC)c1] | 343.1 |
| (R)-mandelic acid 2,3,4- trimethoxyphenylpropanoate | 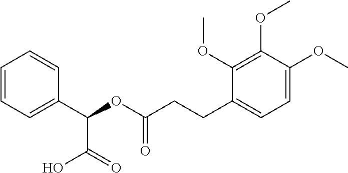 [SMILES: COc1ccc(CCC(=O)O[C@@H](C(=O)O)c2ccccc2)c(OC)c1OC] | 373.1 |
| (R)-mandelic acid 3,4,5- trimethoxyphenylpropanoate | 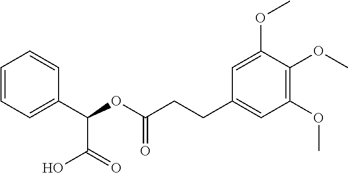 [SMILES: COc1cc(CCC(=O)O[C@@H](C(=O)O)c2ccccc2)cc(OC)c1OC] | 373.1 |
| (R)-mandelic acid 4- ethoxyphenylpropanoate | 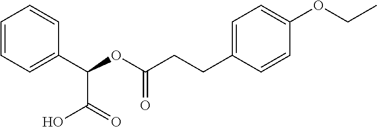 [SMILES: CCOc1ccc(CCC(=O)O[C@@H](C(=O)O)c2ccccc2)cc1] | 327.1 |
| (R)-mandelic acid 4- propoxyphenylpropanoate | 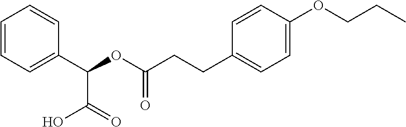 [SMILES: CCCOc1ccc(CCC(=O)O[C@@H](C(=O)O)c2ccccc2)cc1] | 341.1 |
| methyl (R)-mandelate 4-hydroxy- 3-methoxy-phenylpropanoate | 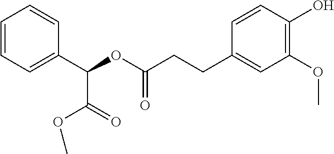 [SMILES: COC(=O)[C@H](OC(=O)CCc1ccc(O)c(OC)c1)c1ccccc1] | 343.1 |
| ethyl (R)-mandelate 4-hydroxy-3- methoxy-phenylpropanoate | 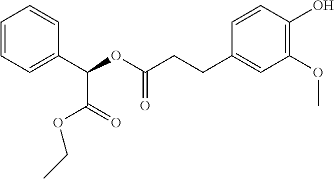 [SMILES: CCOC(=O)[C@H](OC(=O)CCc1ccc(O)c(OC)c1)c1ccccc1] | 357.1 |
| isopropyl (RS)-mandelate 4- hydroxy-3-methoxy- phenylpropanoate | 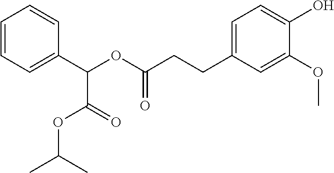 [SMILES: COc1cc(CCC(=O)OC(C(=O)OC(C)C)c2ccccc2)ccc1O] | 371.2 |
| isoamyl (RS)-mandelate 4- hydroxy-3-methoxy- phenylpropanoate | 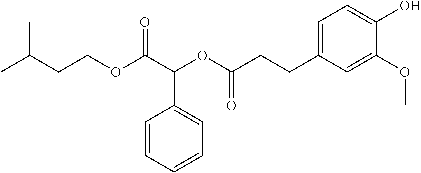 [SMILES: COc1cc(CCC(=O)OC(C(=O)OCCC(C)C)c2ccccc2)ccc1O] | 399.2 |
| (S)-mandelic acid phenylpropanoate | 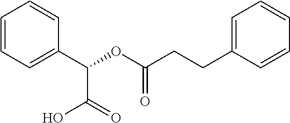 [SMILES: O=C(CCc1ccccc1)O[C@H](C(=O)O)c1ccccc1] | 283.1 |
| (S)-mandelic acid 2-hydroxy- phenylpropanoate | 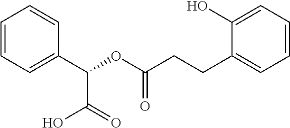 [SMILES: O=C(CCc1ccccc1O)O[C@H](C(=O)O)c1ccccc1] | 299.1 |
| (S)-mandelic acid 3-hydroxy- phenylpropanoate | 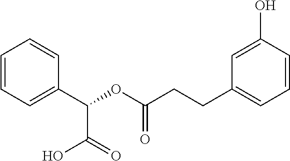 [SMILES: O=C(CCc1cccc(O)c1)O[C@H](C(=O)O)c1ccccc1] | 299.1 |
| (S)-mandelic acid 4-hydroxy- phenylpropanoate | 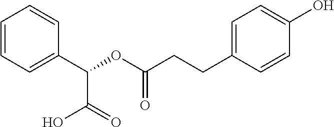 [SMILES: O=C(CCc1ccc(O)cc1)O[C@H](C(=O)O)c1ccccc1] | 299.1 |
| (S)-mandelic acid 3,4- dihydroxyphenylpropanoate | 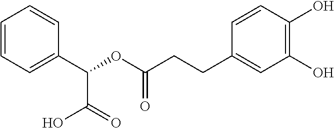 [SMILES: O=C(CCc1ccc(O)c(O)c1)O[C@H](C(=O)O)c1ccccc1] | 315.1 |
| (S)-mandelic acid 2- methoxyphenylpropanoate | 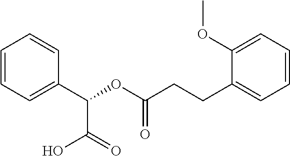 [SMILES: COc1ccccc1CCC(=O)O[C@H](C(=O)O)c1ccccc1] | 313.1 |
| (S)-mandelic acid 3- methoxyphenylpropanoate | 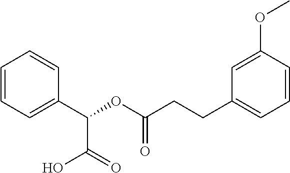 [SMILES: COc1cccc(CCC(=O)O[C@H](C(=O)O)c2ccccc2)c1] | 313.1 |
| (S)-mandelic acid 4- methoxyphenylpropanoate | 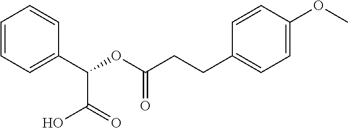 [SMILES: COc1ccc(CCC(=O)O[C@H](C(=O)O)c2ccccc2)cc1] | 313.1 |
| (S)-mandelic acid 4-hydroxy-3- methoxyphenylpropanoate | 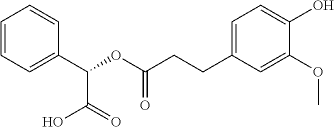 [SMILES: COc1cc(CCC(=O)O[C@H](C(=O)O)c2ccccc2)ccc1O] | 329.1 |
| (S)-mandelic acid 3,5- dimethoxyphenylpropanoate | 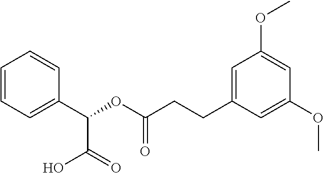 [SMILES: COc1cc(CCC(=O)O[C@H](C(=O)O)c2ccccc2)cc(OC)c1] | 343.1 |
| (S)-mandelic acid 3,4,5- trimethoxyphenylpropanoate | 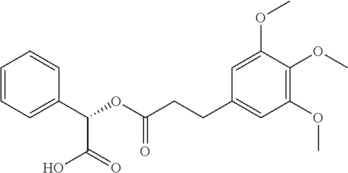 [SMILES: COc1cc(CCC(=O)O[C@H](C(=O)O)c2ccccc2)cc(OC)c1OC] | 373.1 |
| (S)-mandelic acid 4-ethoxy- phenylpropanoate | 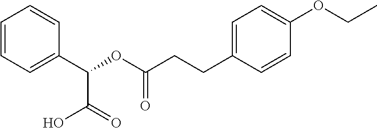 [SMILES: CCOc1ccc(CCC(=O)O[C@H](C(=O)O)c2ccccc2)cc1] | 327.1 |
| (S)-mandelic acid 4-propoxy- phenylpropanoate | 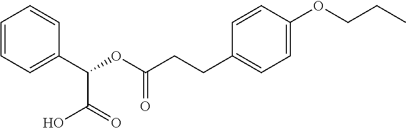 [SMILES: CCCOc1ccc(CCC(=O)O[C@H](C(=O)O)c2ccccc2)cc1] | 341.1 |
| Methyl (S)-mandelate 4-hydroxy- 3-methoxy-phenylpropanoate | 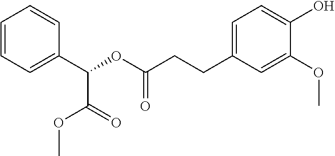 [SMILES: COC(=O)[C@@H](OC(=O)CCc1ccc(O)c(OC)c1)c1ccccc1] | 343.1 |
| Ethyl (S)-mandelate 4-hydroxy-3- methoxy-phenylpropanoate | 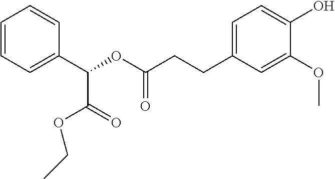 [SMILES: CCOC(=O)[C@@H](OC(=O)CCc1ccc(O)c(OC)c1)c1ccccc1] | 357.1 |
| (RS)-4-hydroxymandelic acid 2- hydroxyphenylpropanoate | 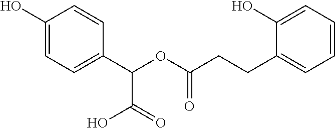 [SMILES: O=C(CCc1ccccc1O)OC(C(=O)O)c1ccc(O)cc1] | 315.1 |
| (RS)-4-hydroxymandelic acid 3- hydroxyphenylpropanoate | 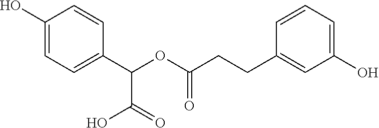 [SMILES: O=C(CCc1cccc(O)c1)OC(C(=O)O)c1ccc(O)cc1] | 315.1 |
| (RS)-4-hydroxymandelic acid 4- hydroxyphenylpropanoate | 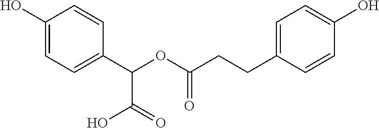 [SMILES: O=C(CCc1ccc(O)cc1)OC(C(=O)O)c1ccc(O)cc1] | 315.1 |
| (RS)-4-hydroxymandelic acid 3,4- dihydroxyphenyl-propanoate | 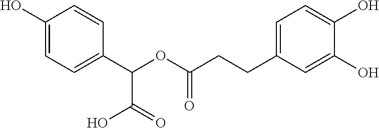 [SMILES: O=C(CCc1ccc(O)c(O)c1)OC(C(=O)O)c1ccc(O)cc1] | 331.1 |
| (RS)-4-hydroxymandelic acid 2- methoxyphenylpropanoate | 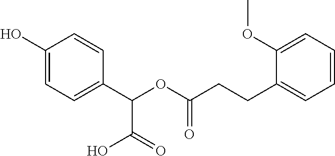 [SMILES: COc1ccccc1CCC(=O)OC(C(=O)O)c1ccc(O)cc1] | 329.1 |
| (RS)-4-hydroxymandelic acid 3- methoxyphenylpropanoate | 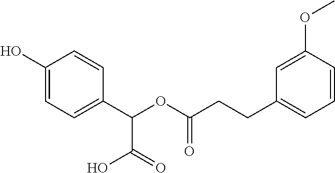 [SMILES: COc1cccc(CCC(=O)OC(C(=O)O)c2ccc(O)cc2)c1] | 329.1 |
| (RS)-4-hydroxymandelic acid 4- methoxyphenylpropanoate | 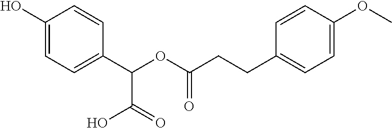 [SMILES: COc1ccc(CCC(=O)OC(C(=O)O)c2ccc(O)cc2)cc1] | 329.1 |
| (RS)-4-hydroxymandelic acid 4- hydroxy-3-methoxyphenyl- propanoate | 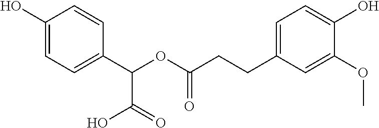 [SMILES: COc1cc(CCC(=O)OC(C(=O)O)c2ccc(O)cc2)ccc1O] | 345.1 |
| (RS)-4-hydroxymandelic acid 3,5- dimethoxyphenyl-propanoate | 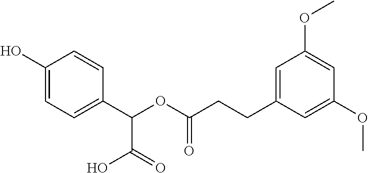 [SMILES: COc1cc(CCC(=O)OC(C(=O)O)c2ccc(O)cc2)cc(OC)c1] | 359.1 |
| (RS)-4-hydroxymandelic acid 4- ethoxyphenylpropanoate | 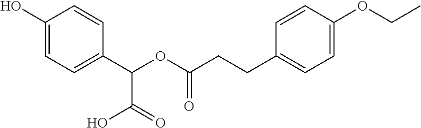 [SMILES: CCOc1ccc(CCC(=O)OC(C(=O)O)c2ccc(O)cc2)cc1] | 343.1 |
| (RS)-4-hydroxymandelic acid 4- propoxyphenylpropanoate | 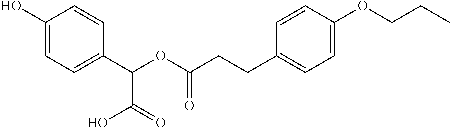 [SMILES: CCCOc1ccc(CCC(=O)OC(C(=O)O)c2ccc(O)cc2)cc1] | 357.1 |
| (RS)-4-methoxymandelic acid 2- hydroxy-phenylpropanoate | 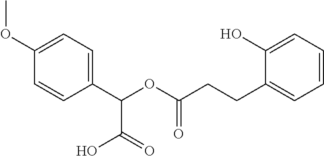 [SMILES: COc1ccc(C(OC(=O)CCc2ccccc2O)C(=O)O)cc1] | 329.1 |
| (RS)-4-methoxymandelic acid 3- hydroxy-phenylpropanoate | 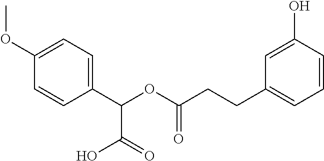 [SMILES: COc1ccc(C(OC(=O)CCc2cccc(O)c2)C(=O)O)cc1] | 329.1 |
| (RS)-4-methoxymandelic acid 4- hydroxyphenyl-propanoate | 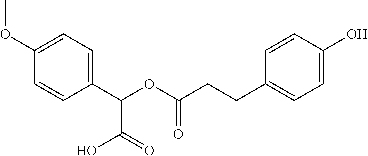 [SMILES: COc1ccc(C(OC(=O)CCc2ccc(O)cc2)C(=O)O)cc1] | 329.1 |
| (RS)-4-methoxymandelic acid 3,4- dihydroxyphenylpropanoate | 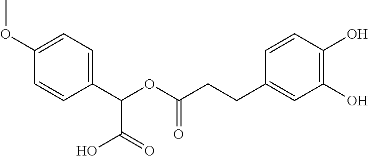 [SMILES: COc1ccc(C(OC(=O)CCc2ccc(O)c(O)c2)C(=O)O)cc1] | 345.1 |
| (RS)-4-methoxymandelic acid 2- methoxyphenylpropanoate | 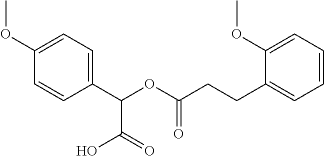 [SMILES: COc1ccc(C(OC(=O)CCc2ccccc2OC)C(=O)O)cc1] | 343.1 |
| (RS)-4-methoxymandelic acid 3- methoxyphenylpropanoate | 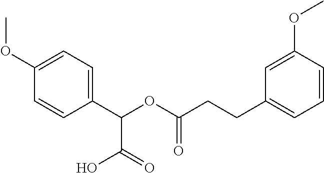 [SMILES: COc1ccc(C(OC(=O)CCc2cccc(OC)c2)C(=O)O)cc1] | 343.1 |
| (RS)-4-methoxymandelic acid 4- methoxyphenylpropanoate | 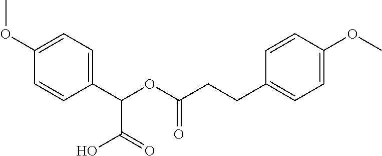 [SMILES: COc1ccc(CCC(=O)OC(C(=O)O)c2ccc(OC)cc2)cc1] | 345.1 |
| (RS)-4-methoxymandelic acid 4- hydroxy-3-methoxyphenyl- propanoate | 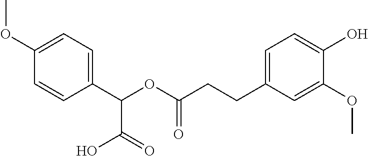 [SMILES: COc1ccc(C(OC(=O)CCc2ccc(O)c(OC)c2)C(=O)O)cc1] | 359.1 |
| (RS)-4-methoxymandelic acid 3,5- dimethoxyphenyl-propanoate | 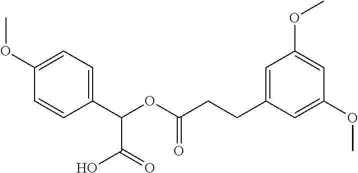 [SMILES: COc1ccc(C(OC(=O)CCc2cc(OC)cc(OC)c2)C(=O)O)cc1] | 373.1 |
| (RS)-4-methoxymandelic acid 4- ethoxyphenylpropanoate | 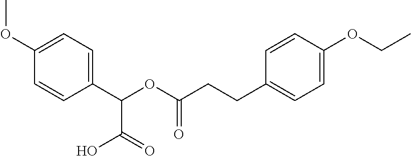 [SMILES: CCOc1ccc(CCC(=O)OC(C(=O)O)c2ccc(OC)cc2)cc1] | 357.1 |
| (RS)-4-methoxymandelic acid 4- propoxyphenylpropanoate | 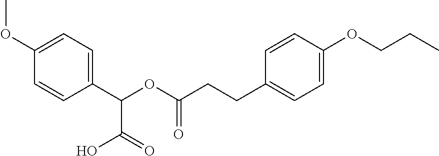 [SMILES: CCCOc1ccc(CCC(=O)OC(C(=O)O)c2ccc(OC)cc2)cc1] | 371.2 |
| (RS)-4-propoxymandelic acid 2- hydroxyphenylpropanoate | 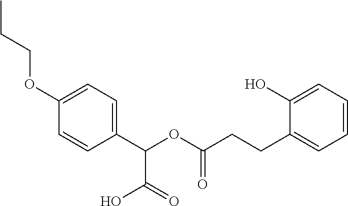 [SMILES: CCCOc1ccc(C(OC(=O)CCc2ccccc2O)C(=O)O)cc1] | 357.1 |
| (RS)-4-propoxymandelic acid 3- hydroxyphenylpropanoate | 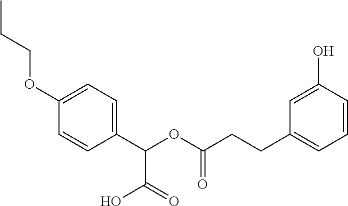 [SMILES: CCCOc1ccc(C(OC(=O)CCc2cccc(O)c2)C(=O)O)cc1] | 357.1 |
| (RS)-4-propoxymandelic acid 4- hydroxyphenyl-propanoate | 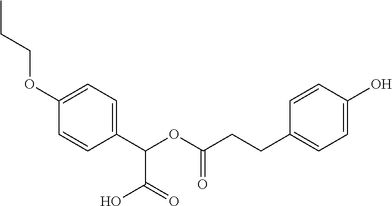 [SMILES: CCCOc1ccc(C(OC(=O)CCc2ccc(O)cc2)C(=O)O)cc1] | 357.1 |
| (RS)-4-propoxymandelic acid 3,4- dihydroxyphenylpropanoate | 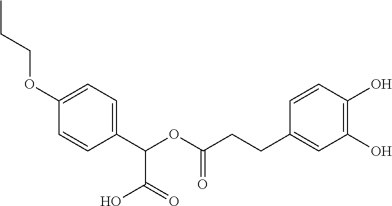 [SMILES: CCCOc1ccc(C(OC(=O)CCc2ccc(O)c(O)c2)C(=O)O)cc1] | 373.1 |
| (RS)-4-propoxymandelic acid 2- methoxyphenylpropanoate | 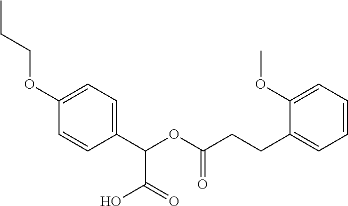 [SMILES: CCCOc1ccc(C(OC(=O)CCc2ccccc2OC)C(=O)O)cc1] | 371.2 |
| (RS)-4-propoxymandelic acid 3- methoxyphenylpropanoate | 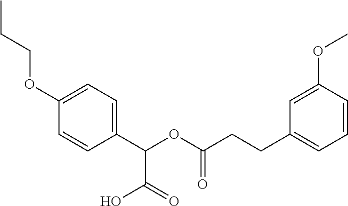 [SMILES: CCCOc1ccc(C(OC(=O)CCc2cccc(OC)c2)C(=O)O)cc1] | 371.2 |
| (RS)-4-propoxymandelic acid 4- methoxyphenylpropanoate | 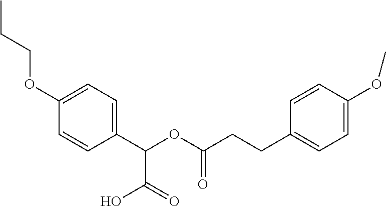 [SMILES: CCCOc1ccc(C(OC(=O)CCc2ccc(OC)cc2)C(=O)O)cc1] | 371.2 |
| (RS)-4-propoxymandelic acid 4- hydroxy-3-methoxyphenyl- propanoate | 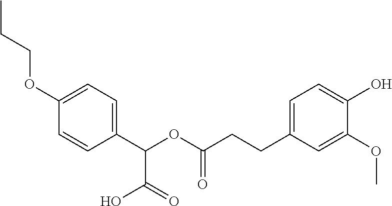 [SMILES: CCCOc1ccc(C(OC(=O)CCc2ccc(O)c(OC)c2)C(=O)O)cc1] | 387.1 |
| (RS)-4-propoxymandelic acid 3,5- dimethoxyphenyl-propanoate | 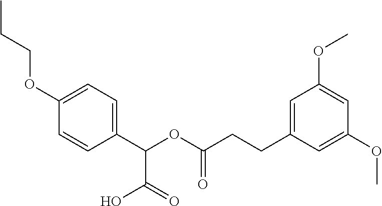 [SMILES: CCCOc1ccc(C(OC(=O)CCc2cc(OC)cc(OC)c2)C(=O)O)cc1] | 401.2 |
| (RS)-4-propoxymandelic acid 4- ethoxyphenylpropanoate | 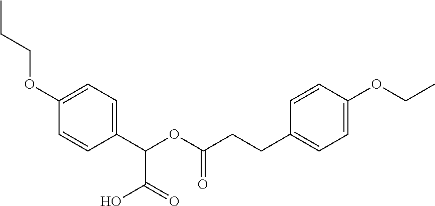 [SMILES: CCCOc1ccc(C(OC(=O)CCc2ccc(OCC)cc2)C(=O)O)cc1] | 385.2 |
| (RS)-4-propoxymandelic acid 4- propoxyphenylpropanoate | 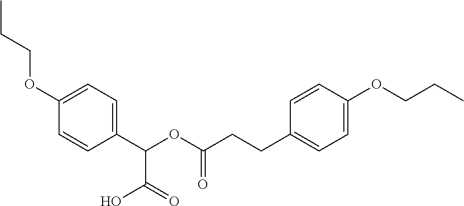 [SMILES: CCCOc1ccc(CCC(=O)OC(C(=O)O)c2ccc(OCCC)cc2)cc1] | 399.2 |
| (RS)-4-hydroxy-3- methoxymandelic acid 2- hydroxyphenylpropanoate | 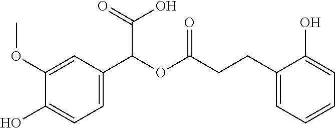 [SMILES: COc1cc(C(OC(=O)CCc2ccccc2O)C(=O)O)ccc1O] | 345.1 |
| (RS)-4-hydroxy-3-methoxy- mandelic acid 3-hydroxy- phenylpropanoate | 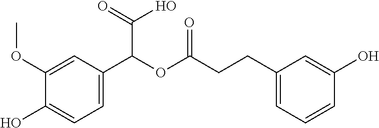 [SMILES: COc1cc(C(OC(=O)CCc2cccc(O)c2)C(=O)O)ccc1O] | 345.1 |
| (RS)-4-hydroxy-3-methoxy- mandelic acid 4-hydroxy- phenylpropanoate | 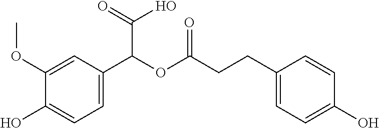 [SMILES: COc1cc(C(OC(=O)CCc2ccc(O)cc2)C(=O)O)ccc1O] | 345.1 |
| (RS)-4-hydroxy-3-methoxy- mandelic acid 3,4-dihydroxy- phenylpropanoate | 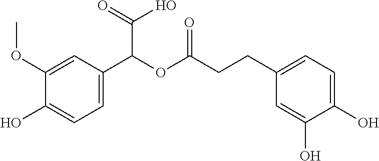 [SMILES: COc1cc(C(OC(=O)CCc2ccc(O)c(O)c2)C(=O)O)ccc1O] | 361.1 |
| (RS)-4-hydroxy-3-methoxy- mandelic acid 2-methoxy- phenylpropanoate | 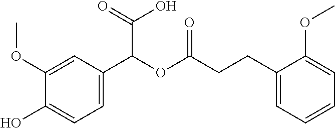 [SMILES: COc1cc(C(OC(=O)CCc2ccccc2OC)C(=O)O)ccc1O] | 359.1 |
| (RS)-4-hydroxy-3-methoxy- mandelic acid 3-methoxy- phenylpropanoate | 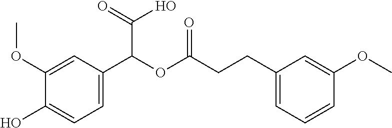 [SMILES: COc1cccc(CCC(=O)OC(C(=O)O)c2ccc(O)c(OC)c2)c1] | 359.1 |
| (RS)-4-hydroxy-3-methoxy- mandelic acid 4-methoxy- phenylpropanoate | 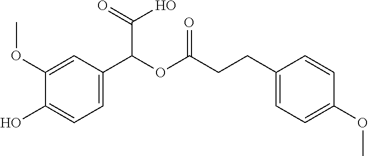 [SMILES: COc1ccc(CCC(=O)OC(C(=O)O)c2ccc(O)c(OC)c2)cc1] | 359.1 |
| (RS)-4-hydroxy-3-methoxy- mandelic acid 4-hydroxy-3- methoxy-phenylpropanoate | 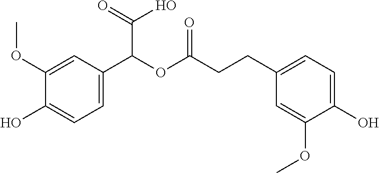 [SMILES: COc1cc(CCC(=O)OC(C(=O)O)c2ccc(O)c(OC)c2)ccc1O] | 375.1 |
| (RS)-4-hydroxy-3-methoxy- mandelic acid 3,5-dimethoxy- phenylpropanoate | 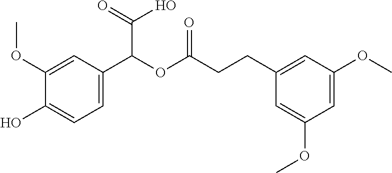 [SMILES: COc1cc(CCC(=O)OC(C(=O)O)c2ccc(O)c(OC)c2)cc(OC)c1] | 389.1 |
| (RS)-4-hydroxy-3-methoxy- mandelic acid 4-ethoxy- phenylpropanoate | 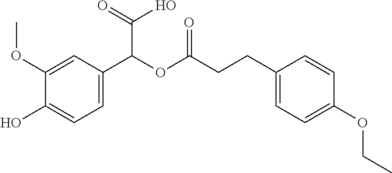 [SMILES: CCOc1ccc(CCC(=O)OC(C(=O)O)c2ccc(O)c(OC)c2)cc1] | 373.1 |
| (RS)-4-hydroxy-3-methoxy- mandelic acid 4-propoxy- phenylpropanoate | 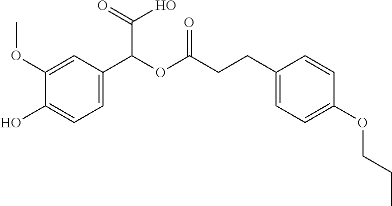 [SMILES: CCCOc1ccc(CCC(=O)OC(C(=O)O)c2ccc(O)c(OC)c2)cc1] | 387.1 |

### Conclusion

Compounds of Formula (I) were successfully synthesized and purified by the described procedure.

### Example 2

### Objective

The objective was to test the modulating effects of compounds of Formula (I) on three pharmacological targets related to cellular senescence.

### Test Compounds

The test compounds prepared in Example 1 were dissolved in DMSO prior to testing.

### Phosphodiesterase 4D (PDE4D)

PDE4D hydrolyzes the secondary messenger, cAMP, which is a regulator and mediator of a number of cellular responses to extracellular signals. PDE4D plays a particularly important role in skin aging, and elevation of cAMP by inhibition of PDE4D holds a potential to alleviate the senescence-associated secretory phenotype (SASP) in the skin.

### Assay

The PDE-Glo™ Phosphodiesterase Assay (Catalog no. V1361) was purchased from Promega (USA). Active PDE4D (Catalog no. P92-31DG-05) was purchased from SignalChem Biotech (Canada). PDE4D stock (0.1 μg/mL) was diluted in assay buffer. The assay was conducted in a 96-well format according to the manufacturer's protocol. All dilutions were performed in assay buffer. Rolipram was used as positive inhibitor control. The procedure was as follows: 10 μl of diluted PDE4D was added to each well followed by 5 μL of test compound solution or vehicle control. The reaction was initiated by adding 10 μL of 2.5 μM cAMP to each well followed by incubation at room temperature for 1 hour. The reaction was terminated by adding 12.5 μL PDE-Glo™ Termination Buffer to each well followed by 12.5 μL PDE-Glo™ Detection Solution. The plate was incubated at room temperature for 20 minutes whereafter 50 μL of the luciferase-based Kinase-Glo™ Reagent was added to each well followed by incubation at room temperature for 10 minutes. The chemiluminescence was measured on a Spectramax ID5 (Molecular Devices) with 1000 ms integration time.

### Results

After subtraction of background controls, the concentration inhibiting the assay by 50% (IC-50) relative to the vehicle control was established for each test compound (see the table below).

| | IC-50 |
|---|---|
| Test Compound | (μg/mL) |
| (R)-mandelic acid 2-hydroxyphenylpropanoate | 0.19 |
| (R)-mandelic acid 3-hydroxyphenylpropanoate | 0.09 |
| (R)-mandelic acid 4-hydroxyphenylpropanoate | 0.11 |
| (R)-mandelic acid 3,4-dihydroxyphenylpropanoate | 0.12 |
| (R)-mandelic acid 3,4,5-trihydroxyphenylpropanoate | 0.17 |
| (R)-mandelic acid 2-methoxyphenylpropanoate | 0.11 |
| (R)-mandelic acid 3-methoxyphenylpropanoate | 0.11 |
| (R)-mandelic acid 4-methoxyphenylpropanoate | 0.20 |
| (R)-mandelic acid 4-hydroxy-3-methoxyphenylpropanoate | 0.09 |
| (R)-mandelic acid 4-hydroxy-3,5-dimethoxyphenylpropanoate | 0.19 |
| (R)-mandelic acid 2,3-dimethoxyphenylpropanoate | 0.86 |
| (R)-mandelic acid 2,4-dimethoxyphenylpropanoate | 0.10 |
| (R)-mandelic acid 2,5-dimethoxyphenylpropanoate | 0.09 |
| (R)-mandelic acid 3,4-dimethoxyphenylpropanoate | 0.20 |
| (R)-mandelic acid 3,5-dimethoxyphenylpropanoate | 0.22 |
| (R)-mandelic acid 2,3,4-trimethoxyphenylpropanoate | 0.21 |
| (R)-mandelic acid 3,4,5-trimethoxyphenylpropanoate | 2.58 |
| (R)-mandelic acid 4-ethoxyphenylpropanoate | 0.16 |
| (R)-mandelic acid 4-propoxyphenylpropanoate | 0.13 |
| (S)-mandelic acid 2-hydroxyphenylpropanoate | 0.18 |
| (S)-mandelic acid 3-hydroxyphenylpropanoate | 0.23 |
| (S)-mandelic acid 4-hydroxyphenylpropanoate | 0.37 |
| (S)-mandelic acid 3,4-dihydroxyphenylpropanoate | 0.24 |
| (S)-mandelic acid 2-methoxyphenylpropanoate | 0.12 |
| (S)-mandelic acid 3-methoxyphenylpropanoate | 0.25 |
| (S)-mandelic acid 4-methoxyphenylpropanoate | 0.20 |
| (S)-mandelic acid 4-hydroxy-3-methoxyphenylpropanoate | 0.14 |
| (S)-mandelic acid 3,5-dimethoxy-phenylpropanoate | 0.11 |
| (S)-mandelic acid 3,4,5-trimethoxyphenylpropanoate | 0.04 |
| (S)-mandelic acid 4-ethoxy-phenylpropanoate | 0.23 |
| (S)-mandelic acid 4-propoxyphenylpropanoate | 0.30 |
| (RS)-4-hydroxymandelic acid 2-hydroxyphenylpropanoate | 0.35 |
| (RS)-4-hydroxymandelic acid 3-hydroxyphenylpropanoate | 0.17 |
| (RS)-4-hydroxymandelic acid 4-hydroxyphenyl-propanoate | 0.18 |
| (RS)-4-hydroxymandelic acid 3,4-dihydroxyphenylpropanoate | 0.11 |
| (RS)-4-hydroxymandelic acid 2-methoxyphenylpropanoate | 0.22 |
| (RS)-4-hydroxymandelic acid 3-methoxyphenylpropanoate | 0.30 |
| (RS)-4-hydroxymandelic acid 4-methoxyphenylpropanoate | 0.39 |
| (RS)-4-hydroxymandelic acid 4-hydroxy-3- | 0.11 |
| methoxyphenylpropanoate | |
| (RS)-4-hydroxymandelic acid 3,5-dimethoxy-phenylpropanoate | 0.37 |
| (RS)-4-hydroxymandelic acid 4-ethoxyphenylpropanoate | 0.63 |
| (RS)-4-hydroxymandelic acid 4-propoxyphenylpropanoate | 7.52 |
| (RS)-4-methoxymandelic acid 2-hydroxyphenylpropanoate | 1.94 |
| (RS)-4-methoxymandelic acid 3-hydroxyphenylpropanoate | 1.01 |
| (RS)-4-methoxymandelic acid 4-hydroxyphenylpropanoate | 0.82 |
| (RS)-4-methoxymandelic acid 3,4-dihydroxyphenylpropanoate | 0.60 |
| (RS)-4-methoxymandelic acid 2-methoxyphenylpropanoate | 0.79 |
| (RS)-4-methoxymandelic acid 3-methoxyphenylpropanoate | 1.17 |
| (RS)-4-methoxymandelic acid 4-methoxyphenylpropanoate | 0.84 |
| (RS)-4-methoxymandelic acid 4-hydroxy-3- | 2.63 |
| methoxyphenylpropanoate | |
| (RS)-4-methoxymandelic acid 3,5-dimethoxyphenylpropanoate | 1.01 |
| (RS)-4-methoxymandelic acid 4-ethoxyphenylpropanoate | 0.65 |
| (RS)-4-methoxymandelic acid 4-propoxyphenylpropanoate | 2.20 |
| (RS)-4-propoxymandelic acid 2-hydroxyphenylpropanoate | 0.71 |
| (RS)-4-propoxymandelic acid 3-hydroxyphenylpropanoate | 0.39 |
| (RS)-4-propoxymandelic acid 4-hydroxyphenylpropanoate | 0.37 |
| (RS)-4-propoxymandelic acid 3,4-dihydroxyphenylpropanoate | 0.86 |
| (RS)-4-propoxymandelic acid 2-methoxyphenylpropanoate | 0.37 |
| (RS)-4-propoxymandelic acid 3-methoxyphenylpropanoate | 0.41 |
| (RS)-4-propoxymandelic acid 4-methoxyphenylpropanoate | 1.49 |
| (RS)-4-propoxymandelic acid 4-hydroxy-3- | 1.10 |
| methoxyphenylpropanoate | |
| (RS)-4-propoxymandelic acid 3,5-dimethoxy-phenylpropanoate | 0.39 |
| (RS)-4-propoxymandelic acid 4-ethoxyphenylpropanoate | 0.78 |
| (RS)-4-propoxymandelic acid 4-propoxyphenylpropanoate | 0.57 |
| (RS)-4-hydroxy-3-methoxymandelic acid 2- | 3.79 |
| hydroxyphenylpropanoate | |
| (RS)-4-hydroxy-3-methoxymandelic acid 3- | 6.24 |
| hydroxyphenylpropanoate | |
| (RS)-4-hydroxy-3-methoxymandelic acid 4- | 2.26 |
| hydroxyphenylpropanoate | |
| (RS)-4-hydroxy-3-methoxymandelic acid 3,4- | 0.52 |
| dihydroxyphenylpropanoate | |
| (RS)-4-hydroxy-3-methoxymandelic acid 2- | 1.99 |
| methoxyphenylpropanoate | |
| (RS)-4-hydroxy-3-methoxymandelic acid 3- | 3.90 |
| methoxyphenylpropanoate | |
| (RS)-4-hydroxy-3-methoxymandelic acid 4- | 0.89 |
| methoxyphenylpropanoate | |
| (RS)-4-hydroxy-3-methoxymandelic acid 4-hydroxy-3-methoxy- | 6.01 |
| phenylpropanoate | |
| (RS)-4-hydroxy-3-methoxymandelic acid 3,5- | 1.03 |
| dimethoxyphenylpropanoate | |
| (RS)-4-hydroxy-3-methoxy-mandelic acid 4- | 0.52 |
| ethoxyphenylpropanoate | |
| (RS)-4-hydroxy-3-methoxymandelic acid 4- | 18.04 |
| propoxyphenylpropanoate | |

### Conclusion

The tested compounds of the invention displayed convincing inhibition of PDE4D confirming the more general finding of efficacy in AI-based (in silico) models of compounds of Formula (I) as described in Example 1.

Histone Acetyl Transferase p300 (p300)

Histone acetyltransferases (HATs) enzymes regulate the acetylation of histone and non-histone proteins. p300 is a transcriptional coactivator that acetylates core histones facilitating chromatin decondensation and recruiting basic RNA polymerase machinery. In addition, many non-histone proteins, such as p53, STATs, and alpha interferon receptor, serve as substrates for p300.

### Assay

The P300 Chemiluminescent Assay Kit Assay Kit (Catalog no. 79705) was purchased from BSP Bioscience Inc (USA). The assay was conducted in a 96-well format according to the manufacturer's protocol. All dilutions were performed in assay buffer. Anacardic acid was used as positive inhibitor control. The procedure was as follows: 5 μL of test compound solution or vehicle control was added to the 96-well plate precoated with histone peptide. 20 μL of p300 solution (0.01 ng/μL) was added to all wells except blanks and negative controls, which were added 20 μL assay buffer. The plate was incubated for 30 minutes at room temperature. 25 μL of Acetyl-CoA substrate solution was added to each well followed by incubation at 30° C. for one hour. Supernatants were removed and the plate was washed three times with 200 μL of TBST buffer followed by addition of 100 μL Blocking Buffer (left 10 minutes) and additional washing three times with 200 μL of TBST buffer. 100 μL diluted primary antibody was added to each well followed by incubation for 1 hour at room temperature. The antibody was specific to the acetylated form of the histone peptide and therefore the binding was proportional to p300 activity.

Supernatants were removed and the plate was washed three times with 200 μL of TBST buffer followed by addition of 100 μL Blocking Buffer (left 10 minutes) and additional washing three times with 200 μL of TBST buffer. 100 μL diluted secondary HRP-labeled antibody was added to each well followed by incubation for 1 hour at room temperature. Supernatants were removed and the plate was washed three times with 200 μL of TBST buffer followed by addition of 100 μL Blocking Buffer (left 10 minutes) and additional washing three times with 200 μL of TBST buffer.

100 μL of a mixture of ELISA ECL Substrate A and B was added to each well and the chemiluminescence was read on a Spectramax ID5 (Molecular Devices) with 1000 ms integration time.

### Results

The concentration inhibiting the assay by 50% (IC-50) relative to the vehicle control was established for each test compound (see the table below).

| Test Compound | IC-50 (μg/mL) |
|---|---|
| (R)-mandelic acid 3,4-dihydroxyphenylpropanoate | 0.10 |
| (R)-mandelic acid 4-hydroxy-3-methoxyphenyl-propanoate | 0.57 |
| (R)-mandelic acid 2-hydroxyphenylpropanoate | 16.18 |
| (R)-mandelic acid 3-hydroxyphenylpropanoate | 20.25 |
| (R)-mandelic acid 4-hydroxyphenylpropanoate | 35.40 |
| (S)-mandelic acid 4-hydroxyphenylpropanoate | 9.30 |
| (RS)-4-methoxymandelic acid 4-hydroxyphenylpropanoate | 1.69 |
| (RS)-4-propoxymandelic acid 4-hydroxyphenylpropanoate | 19.07 |

### Conclusion

The tested compounds displayed convincing inhibition of p300 confirming the more general finding of efficacy in AI-based (in silico) models of compounds of Formula (I) as described in Example 1.

### Kelch-Like ECH-Associated Protein 1 (KEAP1)/Nuclear Factor Erythroid 2-Related Factor 2 (NRF2)

The NRF2 antioxidant response pathway plays an important role in cellular defense and regeneration. NRF2, a basic leucine zipper transcription factor, induces the expression of antioxidant and phase II enzymes by binding to the ARE (antioxidant response element) region of the gene promoter. Under basal conditions, NRF2 is retained in the cytosol by binding to the cytoskeletal protein KEAP1. Upon exposure to oxidative stress or other ARE activators, NRF2 is released from KEAP1 and translocates to the nucleus, where it can bind to the ARE, leading to the expression of antioxidant and phase II enzymes that protect the cell from oxidative damage. Therapeutic agents that release NRF2 by binding to KEAP1 can activate the antioxidant response pathway without oxidative stress.

### Assay

The ARE Reporter—Hep G2 Cell line (Catalog no. 60513), ONE-Step™ Luciferase Assay System (Catalog no. 60690) and Growth Medium 1K (Catalog no. 79533) were purchased from BSP Bioscience (USA). The cells were cultured in Growth Medium 1K according to the manufacturer's protocol until the day of assay. The assay was conducted in a 96-well format according to the manufacturer's protocol. All dilutions were performed in Growth Medium 1K. Methyl fumarate at 80 μM was used as positive control. The procedure was as follows: Hep G2 cells were seeded at a density of 40.000 cells per well into a white clear-bottom 96-well microplate in 45 μL of assay medium. 10 μL of test compound solution or vehicle control was added to each well followed by 18 hours incubation at 37° C., 5% CO2. 100 μl of ONE-Step™ Luciferase Assay reagent was added to each well followed by shaking at room temperature for 15 minutes. The ARE luciferase reporter transcription response was measured as chemiluminescence on a Spectramax ID5 (Molecular Devices) and after subtraction of background controls, the relative ARE expression was calculated.

### Results

After subtraction of background controls, the relative ARE expression was calculated. The concentration inducing 50% enhanced ARE transcription response (EC-50) was established for each test compound (see the table below).

| | EC-50 |
|---|---|
| Test Compound | (μg/mL) |
| (R)-mandelic acid 3,4-dihydroxyphenylpropanoate | 0.34 |
| (R)-mandelic acid 4-hydroxy-3,5-dimethoxyphenylpro- | 4.56 |
| panoate | |
| (RS)-4-hydroxymandelic acid 3,4-dihydroxyphenylpro- | 0.71 |
| panoate | |
| (RS)-4-hydroxymandelic acid 4-hydroxy-3-methoxyphenyl- | 4.69 |
| propanoate | |

### Conclusion

The tested compounds displayed convincing activation of the NRF2 pathway, confirming the more general finding of efficacy in AI-based (in silico) models of compounds of Formula (I) as described in Example 1.

### Example 3

### Objective

Seven studies of intrinsic or extrinsic dermal aging were conducted to establish if (R)-mandelic acid 3,4-dihydroxyphenylpropanoate could counteract the aging senescent phenotype.

### Test Compound

(R)-mandelic acid 3,4-dihydroxyphenylpropanoate was prepared according to Example 1.

### Method

A total of seven studies in three models of dermal aging reflecting intrinsic as well as extrinsic induction of senescence (Hernandez-Segura et al 2018) were employed:
1. Intrinsic Aging in primary human dermal fibroblasts.
   In this intrinsic model, the senescent phenotype was mediated by replicative telomere shortening.
   Cells were cultivated until they stopped proliferating (typically >20 passages), whereafter they acquired a senescent phenotype. These cells were seeded in 6 well plates. Non-senescent cells of the same batch (<10 passages) were seeded as non-senescent controls.
   Senescent cells were incubated 48 hours with concentrations of (R)-mandelic acid 3,4-dihydroxyphenylpropanoate or vehicle, as well as the vehicle-treated non-senescent (normal) control.
   This was followed by 3 hours incubation with resazurin to establish relative cell density and proliferation by measuring fluorescence of collected medium (excitation 550 nm and emission 590 nm).
   The cells were harvested, and gene expression analyzed by Real-Time qRT-PCR.
2. DNA Damage Induced Aging in human dermal fibroblasts (fHDF/TERT166).
   In this extrinsic model, the senescent phenotype was induced with doxorubicin.
   Cells were treated with doxorubicin for 24 hours, followed by 6 days of incubation without doxorubicin, whereafter they acquired a senescent phenotype. These cells were seeded in 6 well plates. Cells of the same batch not treated with doxorubicin were seeded as non-senescent controls.
   Senescent cells were incubated 48 hours with concentrations of (R)-mandelic acid 3,4-dihydroxyphenylpropanoate or vehicle, as well as the vehicle-treated non-senescent (normal) control.
   This was followed by 3 hours incubation with resazurin to establish relative cell density and proliferation by measuring fluorescence of collected medium (excitation 550 nm and emission 590 nm).
   The cells were harvested, and gene expression analyzed by Real-Time qRT-PCR.
3. Oxidative Stress Induced Aginq in human dermal fibroblasts (fHDF/TERT166).
   In this extrinsic model, the senescent phenotype was induced with H₂O₂.
   Cells were treated with bouts of H₂O₂ over 6 days, whereafter they acquired a senescent phenotype. These cells were seeded in 6 well plates. Cells of the same batch not treated with H₂O₂ were seeded as non-senescent controls.
   Senescent cells were incubated 48 hours with concentrations of (R)-mandelic acid 3,4-dihydroxyphenylpropanoate or vehicle, as well as the vehicle-treated non-senescent (normal) control.
   This was followed by 3 hours incubation with resazurin to establish relative cell density and proliferation by measuring fluorescence of collected medium (excitation 550 nm and emission 590 nm).
   The cells were harvested, and gene expression analyzed by Real-Time qRT-PCR.

### Key Comparisons

In each model, the expression of key genes involved in skin aging were measured. All models included:
Normal controls (non-senescent, vehicle treated)
Senescent controls (vehicle treated) and
Senescent cells treated with (R)-mandelic acid 3,4-dihydroxyphenylpropanoate (in vehicle).

This design enabled the following important comparisons:
Senescent controls relative to normal controls to establish if significant induction of aging-related gene expression had occurred.
(R)-mandelic acid 3,4-dihydroxyphenylpropanoate treated cells relative to senescent controls to establish if (R)-mandelic acid 3,4-dihydroxyphenylpropanoate inhibited aging-related gene expression.
(R)-mandelic acid 3,4-dihydroxyphenylpropanoate treated cells relative to normal controls to establish if (R)-mandelic acid 3,4-dihydroxyphenylpropanoate treatment could revert the cells to a non-senescent state.

### Study Protocols

| | |
|---|---|
| Title: | Intrinsic Aging Human Dermal |
| Study Type: | Intrinsic/Replicative Aging |
| Cell Type: | Primary Human Dermal Fibroblasts from adult donor |
| Senescent | Replicative senescence was achieved by cultivation until stop of |
| Cells: | proliferation, typically >20 cycles, depending on donor and confirmed by |
| | morphology and β-galactosidase staining (Hernandez-Segura et al 2018). |
| | Normal (non-senescent) control: Same batch of cells <10 cycles (stored |
| | in freezer at −140° C.) |
| Test | 1) (R)-mandelic acid 3,4-dihydroxyphenylpropanoate 2.0 μg/ml (1000× |
| Articles: | stock in 99.8% ethanol). 2) (R)-mandelic acid 3,4- |
| | dihydroxyphenylpropanoate 8.0 μg/ml (1000× stock in 99.8% ethanol). 3) |
| | Controls (99.8% ethanol) |
| Replicates: | All test articles and controls were tested in 4 biological replicates. |
| Procedure: | Cells were seeded in 6-well plates (Sarstedt article no. 83.3920) at 100.000 |
| | cells/well in 2 ml medium/well. Medium: Gibco ™ DMEM/F-12, GlutaMAX ™ |
| | with 10% Fetal Bovine Serum and 1% Penicillin/Streptomycin. Incubation |
| | at 37° C., 5% CO₂. After incubation overnight, the medium was changed, |
| | and active compounds or vehicle was/were added to all wells. Vehicle |
| | (99.8% ethanol) corresponded to 0.1% of the total volume. After |
| | incubation for 48 hours, the medium was changed and added 200 μl |
| | Resazurin Deep Blue Cell Viability Kit (BioLegend cat. No. 424702) and |
| | incubated for 3 hours, whereafter the medium was collected and |
| | transferred to a black fluorescence 96 well plate (Thermo Scientific |
| | 137101) and fluorescence was measured in a Spectramax ID5 at 550 nm |
| | excitation and 590 nm emission to establish cell viability and proliferation. |
| | Cells were lysed and RNA collected and purified using the NucleoSpin |
| | RNA Plus RNA isolation kit (Macherey-Nagel ™ cat. No. 740990.50) and |
| | following the manufacturer's protocol. RNA amount and sample purity |
| | ratios (A260/A280 nm and A260/A230 nm) were measured using on a |
| | Nanodrop Spectrophotometer. RNA was analyzed by qPCR as described |
| | below. |

| | |
|---|---|
| Title: | DNA Damage Induced Aging Human Dermal |
| Study Type: | Doxorubicin Induced Senescence Extrinsic Aging Model |
| Cell Type: | Human dermal fibroblasts (male, foreskin), hTERT immortalized, primary- |
| | like cells |
| Senescent | The senescent phenotype was induced by incubation for 24 hours with |
| Cells: | 250 nM doxorubicin, followed by 6 days incubation without. Confirmed by |
| | morphology and β-galactosidase staining according to Hernandez-Segura |
| | et al 2018. |
| | Normal (non-senescent) control: hTERT immortalized cells of the same |
| | batch. |
| Test | 1) (R)-mandelic acid 3,4-dihydroxyphenylpropanoate 2.0 μg/ml (1000× |
| Articles: | stock in 99.8% ethanol). 2) (R)-mandelic acid 3,4- |
| | dihydroxyphenylpropanoate 8.0 μg/ml (1000× stock in 99.8% ethanol). 3) |
| | Controls (99.8% ethanol) |
| Replicates: | All test articles and controls were tested in 4 biological replicates. |
| Procedure: | Cells were seeded in 6-well plates (Sarstedt article no. 83.3920) at 100.000 |
| | cells/well in 2 ml medium/well. Medium: Gibco ™ DMEM/F-12, GlutaMAX ™ |
| | with 10% Fetal Bovine Serum, 1% Penicillin/Streptomycin and 100 μg/ml |
| | G418 (InvivoGen, cat. no. ant-gn-5). Incubation at 37º C., 5% CO₂. After |
| | incubation overnight, the medium was changed, and active compounds or |
| | vehicle was/were added to all wells. Vehicle (99.8% ethanol) |
| | corresponded to 0.1% of the total volume. |
| | After incubation for 48 hours, the medium was changed and added 200 μl |
| | Resazurin Deep Blue Cell Viability Kit (BioLegend cat. No. 424702) and |
| | incubated for 3 hours, whereafter the medium was collected and |
| | transferred to a black fluorescence 96 well plate (Thermo Scientific |
| | 137101), and fluorescence was measured in a Spectramax ID5 at 550 nm |
| | excitation and 590 nm emission to establish cell viability and proliferation. |
| | Cells were lysed and RNA collected and purified using the NucleoSpin ® |
| | RNA Plus RNA isolation kit (Macherey-Nagel ™ cat. no. 740990.50) and |
| | following the manufacturer's protocol. RNA amount and sample purity |
| | ratios (A260/A280 nm and A260/A230 nm) were measured using on a |
| | Nanodrop Spectrophotometer. RNA was analyzed by qPCR as described |
| | below. |

| | |
|---|---|
| Title: | Oxidative Stress Induced Aging Human Dermal |
| Study Type: | Hydrogen Peroxide Induced Senescence Extrinsic Aging Model |
| Cell Type: | Human dermal fibroblasts (male, foreskin), hTERT immortalized, primary- |
| | like cells |
| Senescent | The senescent phenotype was induced by incubation for 2 hours with 200 |
| Cells | μM H₂O₂, followed by 48 hours incubation without, which was repeated |
| | twice for a total of three H₂O₂ treatments. Confirmed by morphology and |
| | β-galactosidase staining according to Hernandez-Segura et al 2018. |
| | Normal (non-senescent) control: hTERT immortalized cells of the same |
| | batch. |
| Test | 1) (R)-mandelic acid 3,4-dihydroxyphenylpropanoate 1.0 μg/ml (1000× |
| Articles: | stock in 99.8% ethanol). 2) (R)-mandelic acid 3,4- |
| | dihydroxyphenylpropanoate 2.0 μg/ml (1000× stock in 99.8% ethanol). 3) |
| | (R)-mandelic acid 3,4-dihydroxyphenyl-propanoate 4.0 μg/ml (1000× |
| | stock in 99.8% ethanol). 4) (R)-mandelic acid 3,4- |
| | dihydroxyphenylpropanoate 8.0 μg/ml (1000× stock in 99.8% ethanol). |
| | 5) Controls (99.8% ethanol). |
| Replicates: | All test articles and controls were tested in 4 biological replicates. |
| Procedure: | Cells were seeded in 6-well plates (Sarstedt article no. 83.3920) at 100.000 |
| | cells/well in 2 ml medium/well. Medium: Gibco ™ DMEM/F-12, GlutaMAX ™ |
| | with 10% Fetal Bovine Serum, 1% Penicillin/Streptomycin and 100 μg/ml |
| | G418 (InvivoGen, cat. no. ant-gn-5). Incubation at 37º C., 5% CO₂. After |
| | incubation overnight, the medium was changed, and active compounds or |
| | vehicle was/were added to all wells. Vehicle (99.8% ethanol) |
| | corresponded to 0.1% of the total volume. |
| | After incubation for 48 hours the medium was changed and added 200 μl |
| | Resazurin Deep Blue Cell Viability Kit (BioLegend cat. No. 424702) and |
| | incubated for 3 hours, whereafter the medium was collected and |
| | transferred to a black fluorescence 96 well plate (Thermo Scientific |
| | 137101), and fluorescence was measured in a Spectramax ID5 at 550 nm |
| | excitation and 590 nm emission to establish cell viability and proliferation. |
| | Cells were lysed and RNA collected and purified using the NucleoSpin ® |
| | RNA Plus RNA isolation kit (Macherey-Nagel ™ cat. no. 740990.50) and |
| | following the manufacturer's protocol. RNA amount and sample purity |
| | ratios (A260/A280 nm and A260/A230 nm) were measured using on a |
| | Nanodrop Spectrophotometer. RNA was analyzed by qPCR as described |
| | below. |

Real Time qPCR Protocol

| | |
|---|---|
| DNA | CDNA was synthesized employing the High-Capacity cDNA Reverse |
| Synthesis: | Transcription Kit (ThermoFisher, Cat. no 4368814) following the |
| | manufacturer's protocol. Conducted with a BioRad MasterCycler |
| | epgradient S. |
| qPCR: | The qPCR reaction was conducted using the PerfeCTa ® SYBR ® Green |
| | FastMixes (Quantabio, Cat. No 95072-250) following the manufacturer's |
| | instruction using a total volume of 10 μl for the reaction. The reaction was |
| | conducted in a MIC PCR machine (Biomolecular Systems) using the |
| | following cycling program: Initiation at 95° C. for 3 min followed by 40 |
| | cycles of 95° C. for 10 sec followed by 60° C. for 30 sec. Data were recorded |
| | and processed with the micPCR software v. 2.12.2. |
| | Regarding qPCR calculations and statistics, see section below “Primers”. |
| Primers: | GAPDH (Merck KiCqStart ™ Primers); ACTB (Eurofins Genomics); CDKN1A |
| | (Merck KiCqStart ™ Primers); LMNB1 (Eurofins Genomics); MMP1 (Merck |
| | KiCqStart ™ Primers); MMP3 (Merck KiCqStart ™ Primers); MME (Merck |
| | KiCqStart ™ Primers) |
| | MMP12 (Merck KiCqStart ™ Primers); IL6 (Merck KiCqStart ™ Primers); |
| | COL1A1 (Merck KiCqStart ™ Primers); COL3A1 (Merck KiCqStart ™ |
| | Primers); ELN (Merck KiCqStart ™ Primers); TIMP1 (Merck KiCqStart ™ |
| | Primers); DNMT1 (Merck KiCqStart ™ Primers); SIRT1 (Merck KiCqStart ™ |
| | Primers). |
| qPCR | All qPCR calculations and statistics were conducted with the micPCR |
| Calculations | |
| and | software v. 2.12.2. All calculations of relative gene expression were |
| | conducted using ACTB and GAPDH as reference genes employing the |
| Statistics: | REST method in the micPCR software, including statistical analysis (Pfaffl |
| | 2001; Pfaffl 2007). Other calculations and statistics were conducted |
| | employing Analyse-it Ultimate Edition and Microsoft Excel. |

### Results

The data were examined by gene expression across the 7 dermal studies to get the best overview of the observed effects as well as their reproducibility across models.

In all of the data presented below, the gene expression was normalized to the two reference (housekeeping) genes GAPDH and ACTB.

### Effects on Key Markers of Dermal Aging and Senescence

CDKN1A (p21^{cip1/waf1})

CDKN1A is a well-established marker of senescence in dermal fibroblasts and has even been found to correlate with age in biopsies from young, middle-aged, or old humans (Idda et al 2020).

### Intrinsic Aging Studies

In both Intrinsic Aging studies, CDKN1A was significantly (p<0.01 or p<0.05) overexpressed (2.1- to 5.3-fold) in the senescent control relative to the normal control. In both studies and at both concentrations, (R)-mandelic acid 3,4-dihydroxyphenylpropanoate significantly (p<0.05) reduced the expression of CDKN1A relative to the senescent control, amounting to:

$$- 0.2 3 - to 0. 50 - fold at 2. μ g / ml$$

$$- 0.2 2 - to 0. 54 - fold at 8. μ g / ml$$

All (R)-mandelic acid 3,4-dihydroxyphenylpropanoate treated groups in both studies displayed a CDKN2A expression not significantly different relative to the normal control.

### DNA Damage Induced Aging Studies

In both DNA Damage Induced Aging studies, CDKN1A was significantly (p<0.05) overexpressed (3.9- to 4.7-fold) in the senescent control relative to the normal control. In both studies and at both concentrations, (R)-mandelic acid 3,4-dihydroxyphenylpropanoate significantly (p<0.05) reduced the CDKN1A expression relative to the senescent control, amounting to:

$$- 0.2 9 - to 0. 30 - fold at 2. μ g / ml$$

$$- 0.2 6 - to 0. 36 - fold at 8. μ g / ml$$

All (R)-mandelic acid 3,4-dihydroxyphenylpropanoate treated groups in both studies displayed a CDKN2A expression close to (not significant) or only slightly higher than the normal control.

### Oxidative Stress Induced Aging Studies

In all three Oxidative Stress Inced Aging studies, the expression of CDKN1A was significantly (p<0.01 or p<0.05) enhanced (2.1- to 3.6-fold) in the senescent control relative to the normal control.

In all studies and at all concentrations, (R)-mandelic acid 3,4-dihydroxyphenylpropanoate significantly (p<0.05) reduced the CDKN1A expression relative to the senescent control, amounting to:

$$- 0.47 - fold at 1. μ g / ml$$

$$- 0.29 - to 0.3 - fold at 2. μ g / ml$$

$$- 0.37 - fold at 4. μ g / ml$$

$$- and 0.2 6 - to 0.3 6 - fold at 8. μ g / ml .$$

In two out of three studies, the (R)-mandelic acid 3,4-dihydroxyphenylpropanoate concentrations 2.0 mg/ml and 8.0 μg/ml, resulted in a CDKN1A expression significantly (p<0.01 or p<0.05) decreased relative to the normal control.

### Conclusion CDKN1A

In all three aging models, CDKN1A was significantly overexpressed in the senescent controls confirming the senescent state. Taken together, the data convincingly show that (R)-mandelic acid 3,4-dihydroxyphenylpropanoate reduces CDKN1A expression in senescent dermal fibroblasts to a non-senescent level associated with young skin, independent of the mode of senescence induction, at concentrations achievable with topical administration.

### LMNB1 (Lamin B1)

LMNB1 (Lamin B1) expression was significantly downregulated in senescent dermal fibroblasts and its decrease has even been proposed as a marker to quantify cellular senescence in photo-aged skin (Wang et al 2017). For each study, the relative expression of LMNB1 was displayed separately relative to the senescent and the normal controls, respectively.

### Intrinsic Aging Studies

In both Intrinsic Aging studies, the relative expression of LMNB1 was significantly reduced (0.11- to 0.16-fold) in the senescent control relative to the normal control, while both concentrations of (R)-mandelic acid 3,4-dihydroxyphenylpropanoate enhanced the expression to a level slightly lower than, and not significantly different from, the normal control.

All (R)-mandelic acid 3,4-dihydroxyphenylpropanoate groups in both studies displayed a significantly (p<0.05) enhanced LMNB1 expression relative to the senescent control amounting to:

$$- 5. - to 8.9 - fold at 2. μ g / ml$$

$$- 4.9 - to 7. - fold at 8. μ g / ml$$

### DNA Damage Induced Aging Studies

In both DNA Damage Induced Aging studies, the relative expression of LMNB1 was significantly (p<0.01) reduced (0.026- to 0.029-fold) in the senescent control relative to the normal control.

All (R)-mandelic acid 3,4-dihydroxyphenylpropanoate treated groups in both studies displayed a significantly (p<0.05) enhanced LMNB1 expression relative to the senescent control amounting to:

$$- 25.6 - to 27.4 - fold at 2. μ g / ml$$

$$- 23.3 - to 30.3 - fold at 8. μ g / ml$$

Both concentrations of (R)-mandelic acid 3,4-dihydroxyphenylpropanoate enhanced the LMNB1 expression to a level close to the normal control, as only two out of four (R)-mandelic acid 3,4-dihydroxyphenylpropanoate treated groups were significantly (p<0.05) lower relative to the normal control.

### Oxidative Stress Induced Aging Studies

In all three Oxidative Stress Induced Aging studies, the relative expression of LMNB1 was significantly (p>0.05) reduced (0.27- to 0.41-fold) in the senescent control relative to the normal control, while all tested concentrations of (R)-mandelic acid 3,4-dihydroxyphenylpropanoate enhanced the expression to a level close to the normal control or significantly (p<0.05) higher.

All (R)-mandelic acid 3,4-dihydroxyphenylpropanoate concentrations in all three studies displayed a significantly (p<0.05) higher LMNB1 expression relative to the senescent control amounting to:

$$- 1.7 - fold at 1. μ g / ml$$

$$- 2.8 - to 4. - fold at 2. μ g / ml$$

$$- 2.3 - fold at 4. μ g / ml$$

$$- 3. - to 4. - fold at 8. μ g / ml$$

### Conclusion LMNB1

In all three aging models, LMNB1 was significantly downregulated in the senescent controls confirming the senescent state. Taken together, the data convincingly show that (R)-mandelic acid 3,4-dihydroxyphenylpropanoate treatment enhances LMNB1 expression in senescent dermal fibroblasts to a non-senescent level associated with young skin, independent of the mode of senescence induction, at concentrations achievable with topical administration.

### Effects on Key Markers of Dermal Connective Tissue

### Collagen Expression (COL1A1 and COL3A1)

Collagen comprises approximately 70-80% of the dry weight of the dermis and is composed of approximately 80% type I collagen and 15% type III (Waller and Maibach 2006).

Decreased collagen expression plays a central role in the structural changes associated with skin aging and is linked to fibroblast senescence (Ezure et al 2019). This is reflected in a lower expression of Collagen Type I Alpha 1 Chain (COL1A1) and Collagen Type III Alpha 1 Chain (COL3A1).

For each study, the relative expression of COL1A1 and COL3A1 is displayed separately relative to the senescent and the normal controls, respectively.

### Intrinsic Aging Studies (Only COL1A1 Measured)

In both Intrinsic Aging studies, COL1A1 was significantly (p<0.01) underexpressed (0.16- to 0.36-fold) relative to the normal control.

In both studies, both concentrations of (R)-mandelic acid 3,4-dihydroxyphenylpropanoate significantly (p<0.01 or p<0.05) increased COL1A1 expression relative to the senescent control amounting to:

$$- 3.1 - fold at 2 , 0 μ g / ml$$

$$- 7.1 - fold at 8 , 0 μ g / ml$$

In both studies, the highest concentration of (R)-mandelic acid 3,4-dihydroxyphenylpropanoate displayed significantly (p<0.05) enhanced COL1A1 expression relative to the normal control.

### DNA Damage Induced Aging Studies

In both DNA Damage Induced Aging studies, COL3A1 was significantly (p<0.01 or p<0.05) underexpressed (0.17- to 0.29-fold) in the senescent controls relative to the normal controls, while COL1A1 was only significantly (p<0.01) underexpressed (0.40-fold) in one study.

In both studies and at both concentrations, (R)-mandelic acid 3,4-dihydroxyphenylpropanoate treated groups displayed a significantly (p<0.05) enhanced expression of COL1A1 relative to the senescent control amounting to:

$$- 4.3 - to 9.3 - fold at 2. μ g / ml$$

$$- 5. - fold to 25.3 - fold at 8. μ g / ml$$

Furthermore, both concentrations in both studies, displayed a significantly (p<0.05) enhanced COL1A1 expression relative to the normal controls amounting to:

$$- 1.7 - to 8.5 - fold at 2. μ g / ml$$

$$- 2. - fold to 23.2 - fold at 8. μ g / ml$$

In both studies and at both concentrations, (R)-mandelic acid 3,4-dihydroxyphenylpropanoate treated groups displayed a significantly (p<0.05) enhanced expression of COL3A1 relative to the senescent controls, amounting to:

$$- 2.8 - to 8.5 - fold at 2. μ g / ml$$

$$- 3.7 - to 8.5 - fold at 8. μ g / ml$$

3 out of 4 (R)-mandelic acid 3,4-dihydroxyphenylpropanoate treated groups in the two studies displayed significantly (p<0.05) enhanced COL3A1 expression relative to the normal control.

### Oxidative Stress Induced Aging

In all Oxidative Stress Induced Aging studies, COL1A1 was significantly (p<0.05) underexpressed (0.14-0.52-fold) in the senescent controls relative to the normal controls.

In all studies and at all concentrations, (R)-mandelic acid 3,4-dihydroxyphenylpropanoate treated groups displayed a significantly (p<0.05) enhanced COL1A1 expression relative to the senescent controls, amounting to:

$$- 4.1 - to 5.7 - fold at 2. μ g / ml$$

$$- 2.8 - to 13.3 - fold at 8. μ g / ml$$

6 out of 8 (R)-mandelic acid 3,4-dihydroxyphenylpropanoate treated groups in the three studies displayed significantly (p<0.05) enhanced COL1A1 expression relative to the normal control.

In all Oxidative Stress Induced Aging studies, COL3A1 was significantly (p<0.05) underexpressed (0.17-0.42-fold) in the senescent controls relative to the normal controls.

In all studies and at all concentrations, (R)-mandelic acid 3,4-dihydroxyphenylpropanoate treated groups displayed a significantly (p<0.05) enhanced expression of COL3A1 relative to the senescent controls, amounting to:

$$- 4.4 - to 6.2 - fold at 2. μ g / ml$$

$$- 2.8 - to 6.5 - fold at 8. μ g / ml$$

4 out of 8 (R)-mandelic acid 3,4-dihydroxyphenylpropanoate treated groups in the three studies displayed significantly (0<0.01 or p<0.05) enhanced COL3A1 expression relative to the normal control.

### Conclusion COL1A1 and COL3A1

In all seven studies and at all tested concentrations, (R)-mandelic acid 3,4-dihydroxyphenylpropanoate significantly (p<0.01 or p<0.05) enhanced the expression of both COL1A1 and COL3A1 relative to the senescent control, reaching a level, which was similar to the normal control or in many cases higher. This convincingly demonstrates that (R)-mandelic acid 3,4-dihydroxyphenylpropanoate can abolish the aging-related down-regulation of collagen expression at concentrations achievable with topical treatment.

### Elastin (ELN) and TIMP Metallopeptidase Inhibitor 1 (TIMP1)

ELN plays a major structural role in the dermis and is crucial for the elasticity of the dermis. ELN is significantly downregulated in skin aging (Ezure et al 2019; Imokawa et al 2015).

“Tissue Inhibitor of Metalloproteinases 1” (TIMP1) exhibits strong inhibition of matrix metallopeptidases and is released by fibroblasts to control the inflammatory degradation by MMPs. TIMP1 is an essential protector of the dermal connective tissue and the expression of TIMP1 is decreased with fibroblast senescence, both ex vivo and in vivo, thus contributing to increased dermal connective degradation with aging (Hornebeck et al. 2003).

### Intrinsic Aging Studies (Only ELN Measured)

In both Intrinsic Aging studies, ELN was significantly (p<0.05) underexpressed (0.24- to 0.38-fold) in the senescent controls relative to the normal controls.

In both studies and at both concentrations, (R)-mandelic acid 3,4-dihydroxyphenylpropanoate significantly (p<0.01 or p<0.05) increased ELN expression relative to the senescent controls, amounting to:

$$- 2.4 - to 4.8 - fold at 2. μ g / ml$$

$$- 3.5 - to 3.9 - fold at 8. μ g / ml$$

The ELN expression of (R)-mandelic acid 3,4-dihydroxyphenylpropanoate treated groups displayed no statistical significance relative to the normal control in any of the studies.

### DNA Damage Induced Aging Studies

In one study, there was a tendency towards ELN underexpression (0,59-fold) in the senescent control relative to the normal control, but it did not reach statistical significance (p=0.078). In the other study, ELN was significantly (p<0.01) underexpressed (0.41-fold) in the senescent control relative to the normal control.

In both studies and at both concentrations, (R)-mandelic acid 3,4-dihydroxyphenylpropanoate displayed a significantly (p<0.01 or p<0.05) enhanced ELN expression relative to the senescent controls, amounting to:

$$5.4 - fold to 13.4 - fold at 2. μg / ml$$

$$4.5 - fold to 32.1 - fold at 8. μg / ml$$

Furthermore, in both studies, all (R)-mandelic acid 3,4-dihydroxyphenylpropanoate groups displayed significantly (p<0.05) enhanced ELN expression relative to the normal controls.

In one study, TIMP1 was significantly (p<0.01) underexpressed (0.87-fold) in the senescent control relative to the normal control, while expression in the other study was not significantly different.

In both studies and at both concentrations, (R)-mandelic acid 3,4-dihydroxyphenylpropanoate significantly (p<0.01 or p<0.05) increased TIMP1 expression relative to the senescent control, amounting to:

$$1.6 - fold to 4.8 - fold at 2. μg / ml$$

$$1.9 - fold to 7.3 - fold at 8. μg / ml$$

In both studies, all (R)-mandelic acid 3,4-dihydroxyphenylpropanoate groups displayed significantly (p<0.05) enhanced TIMP1 expression relative to the normal controls.

### Oxidative Stress Induced Aging

In all Oxidative Stress Induced Aging studies, ELN was significantly (p<0.05) underexpressed (0.24- to 0.55-fold) in the senescent controls relative to the normal controls.

In all three studies and at all concentrations, (R)-mandelic acid 3,4-dihydroxyphenylpropanoate displayed a significantly (p<0.05) enhanced expression of ELN relative to the senescent controls amounting to:

$$1.9 - fold at 1. μg / ml$$

$$3.6 - fold to 6.1 - fold at 2. μg / ml$$

$$3.7 - fold at 4. μg / ml$$

$$4.6 - fold to 8.7 - fold at 8. μg / ml$$

Furthermore, in 1 out of 3 studies, (R)-mandelic acid 3,4-dihydroxyphenylpropanoate at 2.0 and 8.0 μg/ml displayed a significantly enhanced expression of ELN compared to the normal control.

TIMP1 expression in the senescent control was only significantly (p<0.05) underexpressed (0.55-fold) in 1 out of 3 Oxidative Stress Induced Aging studies.

In all studies, (R)-mandelic acid 3,4-dihydroxyphenylpropanoate at 2.0 μg/ml displayed a significantly (p<0.05) enhanced TIMP1 expression relative to the senescent control at either 2.0 or 8.0 μg/ml.

### Conclusion ELN and TIMP1

In all seven studies and at all tested concentrations, (R)-mandelic acid 3,4-dihydroxyphenylpropanoate significantly (p<0.01 or p<0.05) enhanced the expression of ELN relative to the senescent control, reaching a level which was similar to the normal control or significantly (p<0.05) higher.

This convincingly demonstrates that (R)-mandelic acid 3,4-dihydroxyphenylpropanoate can abolish the aging-related down-regulation of ELN expression at concentrations achievable with topical administration.

Furthermore, in all five studies where TIMP1 was tested and at most tested concentrations, (R)-mandelic acid 3,4-dihydroxyphenylpropanoate significantly (p<0.01 or p<0.05) enhanced the expression of TIMP1 relative to the senescent control, and in four out of five studies reached a level significantly (p<0.05) enhanced relative to the normal control. This effect may contribute to the antiaging effect of (R)-mandelic acid 3,4-dihydroxyphenylpropanoate, as TIMP1 inhibits matrix metallopeptidases.

### Effects on Key Markers of Dermal SASP

The senescence associated secretory phenotype (SASP) is a characteristic feature of dermal aging and has a potential detrimental effect on the connective tissue structure due to the enhanced expression of matrix metallopeptidases.

### Matrix Metallopeptidases MMP1 and MMP3

MMP1 and MMP3 are both overexpressed in aging skin as well as senescent fibroblasts and are implicated in the aging-related dermal connective tissue degradation (Hornebeck et al., 2003).

For each study, the relative expression of MMP1 and MMP3 was displayed separately relative to the senescent and the normal controls, respectively.

### Intrinsic Aging Studies (Only MMP1 Measured)

In both Intrinsic Aging studies, the expression of MMP1 was significantly (p>0.05) enhanced (2.2- to 2.6-fold) in the senescent controls, relative to the normal controls.

All (R)-mandelic acid 3,4-dihydroxyphenylpropanoate concentrations in both studies displayed a significantly (p<0.05) suppressed MMP1 expression relative to the senescent control amounting to:

$$0.39 - to 0.42 - fold at 2. μg / ml$$

$$0.4 - to 0.52 - fold at 8. μg / ml$$

In both studies and at both concentrations, (R)-mandelic acid 3,4-dihydroxyphenylpropanoate displayed MMP 1 expression at a similar level relative to the normal controls (no statistical significance).

### DNA Damage Induced Aging Studies

In both DNA Damage Induced Aging studies, a significantly (p<0.05) enhanced expression was observed of MMP1 (3.0- to 3.2-fold) and MMP3 (2.3-fold and 4.8-fold) in the senescent controls relative to the normal controls.

In both studies and at both concentrations, (R)-mandelic acid 3,4-dihydroxyphenylpropanoate elicited a significantly suppressed expression of MMP1 relative to the senescent controls amounting to:

$$0.17 - to 0.49 - fold at 2. μg / ml$$

$$0.18 - to 0.6 - fold at 8. μg / ml$$

In both studies and at both concentrations, (R)-mandelic acid 3,4-dihydroxyphenylpropanoate elicited a significantly suppressed expression of MMP3 relative to the senescent controls amounting to:

$$0.25 - to 0.32 - fold at 2. μg / ml$$

$$0.23 - to 0.32 - fold at 8. μg / ml$$

In both studies and at both concentrations, (R)-mandelic acid 3,4-dihydroxyphenylpropanoate elicited an MMP1 and MMP3 expression close to the level of the normal control and reaching a significantly (p<0.01 or p<0.05) lower expression in one study.

### Oxidative Stress Induced Aging Studies

In all Oxidative Stress Induced Aging models, a significantly (p<0.01 or p<0.05) enhanced expression was observed of MMP1 (2.7- to 38.3-fold) and MMP3 (4.9- to 8.2-fold) in the senescent controls relative to the normal controls, while (R)-mandelic acid 3,4-dihydroxyphenylpropanoate treated groups displayed an expression level close to the normal controls.

With only one exception, all concentrations of (R)-mandelic acid 3,4-dihydroxyphenylpropanoate in all studies, elicited a significantly (p<0.01 or p<0.05) suppressed expression of MMP1 relative to the senescent controls amounting to:

$$0.04 - fold at 1. μg / ml$$

$$0.03 - to 0.39 - fold at 2. μg / ml$$

$$0.06 - fold at 4. μg / ml$$

$$0.04 - to 0.83 - fold at 8. μg / ml$$

In all studies and at all concentrations, (R)-mandelic acid 3,4-dihydroxyphenylpropanoate elicited a significantly (p<0.01 or p<0.05) suppressed expression of MMP3 relative to the senescent controls amounting to:

$$0.36 - fold at 1. μg / ml$$

$$0.16 - to 0.46 - fold at 2. μg / ml$$

$$0.23 - fold at 4. μg / ml$$

$$0.17 - to 0.18 - fold at 8. μg / ml$$

### Conclusion Matrix Metallopeptidases

In conclusion, the data convincingly show that (R)-mandelic acid 3,4-dihydroxyphenylpropanoate reduces the expression of the key SASP genes MMP1 and MMP3 in senescent dermal fibroblasts, independent of the mode of aging induction, at concentrations that can be achieved with topical administration.

(R)-mandelic acid 3,4-dihydroxyphenylpropanoate treatment was able to reduce the expression to a level which was not different from the normal control in 4 out of 7 studies for MMP1 and 2 out of 5 studies for MMP3.

### Effects on Key Markers of Epigenetic Aina Repair

### DNMT1 (DNA Methyltransferase 1) and SIRT1 (Sirtuin 1)

Lower expression of DNA methyltransferase 1 (DNMT1) is characteristic for senescent dermal fibroblasts. DNMT1 is known as “maintenance DNMT” since it preserves the original non-senescent methylation of dermal cells. DNMT1 expression inversely correlates with chronological age in human skin (Orioli et al 2018).

The class III histone deacetylase SIRT1 exerts control of energy metabolism, inflammation, and oxidative stress, as well as mediating cell survival, UV damage response, DNA repair and tissue regeneration (Garcia-Peterson et al 2017). In dermal fibroblasts, SIRT1 expression is significantly reduced with age (Tigges et al 2014; Carlomosti et al 2017) and, notably, SIRT1 up-regulation or down-regulation results in delayed or accelerated fibroblast senescence, respectively (De Cabo et al 2015).

### Intrinsic Aging Studies

In both Intrinsic Aging studies, DNMT1 was significantly (p<0.01 or p<0.05) underexpressed (0.39- to 0.51-fold) in the senescent controls relative to the normal controls. In both studies and at both concentrations, (R)-mandelic acid 3,4-dihydroxyphenylpropanoate significantly (p>0.001, p<0.01 or p<0.05) increased DNMT1 expression relative to the senescent control, amounting to:

$$2.7 - to 2.9 - fold at 2. μg / ml$$

$$2.8 - to 2.9 - fold at 8. μg / ml$$

Furthermore, (R)-mandelic acid 3,4-dihydroxyphenylpropanoate at 2.0 and 8.0 μg/ml elicited a significantly (p<0.01 or p<0.05) enhanced DNMT1 expression relative to the normal control in one study and was not different from the control in the other study.

In both Intrinsic Aging studies, SIRT1 was significantly (p<0.05) underexpressed (0.53- to 0.64-fold) in the senescent controls relative to the normal controls.

In both studies and at both concentrations, (R)-mandelic acid 3,4-dihydroxyphenylpropanoate significantly (p<0.01 or p<0.05) increased SIRT1 expression relative to the senescent control, amounting to:

$$2.8 - to 3. , 2 - fold at 2. μg / ml$$

$$2. , 8 - to 3.4 - fold at 8. , 0 μg / ml$$

Furthermore, (R)-mandelic acid 3,4-dihydroxyphenylpropanoate at 2.0 and 8.0 μg/ml (R)-mandelic acid 3,4-dihydroxyphenylpropanoate displayed a significantly (p>0.001, p<0.01 or p<0.05) enhanced expression of DNMT1 relative to the normal controls in both studies, amounting to:

$$1.7 - to 1.8 - fold at 2. μg / ml$$

$$1.8 - to 1.8 - fold at 8. μg / ml$$

### DNA Damage Induced Aging Studies

In both DNA Damage Induced Aging studies, DNMT1 was significantly (p<0.01 or p<0.05) underexpressed (0.11- to 0.32-fold) in the senescent controls relative to the normal controls.

In both studies and at both concentrations, (R)-mandelic acid 3,4-dihydroxyphenylpropanoate significantly (p<0.01 or p<0.05) increased DNMT1 expression relative to the senescent controls, amounting to:

$$4.6 - to 5.2 - fold at 2. μg / ml$$

$$5. - to 5.6 - fold at 8. μg / ml$$

In one study, the highest concentration of (R)-mandelic acid 3,4-dihydroxyphenylpropanoate displayed a significantly (p<0.05) enhanced DNMT1 expression relative to the normal control, while both concentrations were significantly (p<0.01 or p<0.05) lower in the other study.

In both DNA Damage Induced Aging studies, SIRT1 was significantly (p<0.01 or p<0.05) underexpressed (0.20- to 0.54-fold) in the senescent controls relative to the normal controls.

In both studies and at both concentrations, (R)-mandelic acid 3,4-dihydroxyphenylpropanoate significantly (p<0.05) increased SIRT1 expression relative to the senescent controls, amounting to:

$$3.2 - to 3.5 - fold at 2. μg / ml$$

$$2.8 - to 3.1 - fold at 8. μg / ml$$

In one study, the highest concentration of (R)-mandelic acid 3,4-dihydroxyphenylpropanoate displayed a significantly (p<0.05) enhanced SIRT1 expression relative to the normal control, while both concentrations were significantly (p<0.05) lower in the other study.

### Oxidative Stress Induced Aging Studies

In 2 out of 3 Oxidative Stress Induced Aging studies, DNMT1 was significantly (p<0.05) underexpressed (0.50- to 0.84-fold) in the senescent controls relative to the normal controls.

In all three studies, (R)-mandelic acid 3,4-dihydroxyphenylpropanoate at 2.0 μg/ml and 8.0 μg/ml significantly (p<0.05) increased DNMT1 expression relative to the senescent controls, amounting to:

$$1.5 - to 1.9 - fold at 2. μg / ml$$

$$1.4 - to 2.9 - fold at 8. μg / ml$$

Furthermore, (R)-mandelic acid 3,4-dihydroxyphenylpropanoate at 2.0 μg/ml and 8.0 μg/ml elicited a significantly (p<0.05) enhanced expression of DNMT1 compared to the normal control in 2 out of 3 studies.

SIRT1 expression displayed a tendency towards suppression (0.58- to 0.89-fold) in the senescent controls relative to the normal controls, which was statistically significant (p<0.05) in only one study.

In all three studies, (R)-mandelic acid 3,4-dihydroxyphenylpropanoate at 2.0 μg/ml and 8.0 μg/ml significantly (p<0.05) increased SIRT1 expression relative to the senescent controls, amounting to:

$$2.1 - to 3.5 - fold at 2. μg / ml$$

$$1.8 - to 4.2 - fold at 8. μg / ml$$

Furthermore, (R)-mandelic acid 3,4-dihydroxyphenylpropanoate at 2.0 μg/ml and 8.0 μg/ml displayed a significantly (p<0.05) enhanced expression of SIRT1 compared to the normal controls in all three studies.

### Conclusion DNMT1 and SIRT1

In all seven studies across the three dermal aging models, (R)-mandelic acid 3,4-dihydroxyphenyl-propanoate treatment significantly and substantially enhanced the expression of DNMT1 and SIRT1 relative to the senescent controls, reaching the level of the normal control or significantly higher.

This convincingly demonstrates that (R)-mandelic acid 3,4-dihydroxyphenylpropanoate induces epigenetic repair pathways, thus eliminating their aging-related downregulation at concentrations achievable with topical administration.

### Collective Conclusion on Dermal Aging Studies

All of the seven studies, comprising the three dermal aging models, displayed the key elements of an aging senescent phenotype in accordance with the changes seen in the aging human dermis:
Significant and substantial increase in the expression of the cell cycle arrest mediating cyclin dependent kinase inhibitor p21^{cip1/waf1} (CDKN1A).
Significant and substantial decrease in the expression of the nuclear lamina protein Lamin B1 (LMNB1) which is essential for nuclear stability.
Significant and substantial decrease in the expression of essential connective tissue components including proteins related to collagen I (COL1A1), collagen III (COL3A1) and elastin (ELN) which are essential for maintaining the thickness, structure and elasticity of young skin.
Significant and substantial increase in the expression of SASP-related matrix metallopeptidases including MMP1 and MMP3 which cause aging-related degradation and damage to the dermal connective tissue.
Significant and substantial decrease in the expression of essential epigenetic maintenance and repair genes including the class III histone deacetylase sirtuin 1 (SIRT1) and DNA methyltransferase 1 (DNMT1).

In all seven studies, (R)-mandelic acid 3,4-dihydroxyphenylpropanoate treatment displayed a significant, reproducible, and substantial reversing effect on all of the above traits of dermal aging leading to a non-senescent phenotype associated with younger skin.

Based on the data, we conclude that (R)-mandelic acid 3,4-dihydroxyphenylpropanoate is effective at the concentrations achievable in the dermis with a topical formulation.

### Example 4

### Objective

Two studies of aging- and cancer-related DNA damage in human skin were conducted to establish if compounds of the invention can enhance the repair of DNA damage induced by ultraviolet (UV) light exposure.

### Test Compound

(R)-mandelic acid 4-hydroxy-3-methoxyphenylpropanoate was prepared according to Example 1.

### Method

DNA damage is a major driver of cellular senescence and aging. Insufficient repair of DNA damage may furthermore lead to mutations and cancer development. The capacity to repair DNA damage gradually declines with age and this decline plays a significant role in the acceleration of skin aging and the risk of skin cancer.

Absorption of ultraviolet (UV) light produces two predominant types of DNA damage, cyclobutane pyrimidine dimers (CPDs) and pyrimidine (6-4) pyrimidone photoproducts which play a central role in skin aging and the development of human skin cancers. UV damaged DNA is usually repaired by nucleotide excision repair or base excision repair. After UV exposure, cells stall the cell cycle for repair. Insufficient DNA repair capacity plays an essential role in skin aging and development of the senescent phenotype, hence the term “photoaging”.

Two different models of UV-induced DNA damage were employed and, in both models, the treatment with (R)-mandelic acid 4-hydroxy-3-methoxyphenyl-propanoate was initiated after the induction of DNA damage to clarify if (R)-mandelic acid 4-hydroxy-3-methoxyphenyl-propanoate could accelerate the DNA repair.

1. UV-Induced DNA Damage in Human Skin Explants from a Middle-Aged Donor.
In this model, 11 mm circular skin biopsies from the same donor (female, age 52 years) were provided by Genoskin, France. The explants (Nativeskin Access®) were embedded in a matrix that maintained all normal skin physiology for up to 7 days and allowed for cultivation in a nutrient medium in 12-well plates in an incubator at 37° C. and 5% CO₂.
Significant DNA damage was induced by placing the explants in a UV-solar simulator (SOL 500 with UV-filter H2) from Dr. Hönle GmbH, Germany. This provided a combination of UVA and UVB comparable to natural sunlight.
3 groups of 4 explants were exposed to 100 mJ/cm² UVB once daily for two consecutive days. A control group of 4 explants without UV exposure was included to estimate the increase in DNA damage caused by the UV treatment.
Immediately after the last UV-treatment, the groups of explants were treated topically on the surface of the skin with 10 μL/explant of one of the following:
   4.0% (w/w) (R)-mandelic acid 4-hydroxy-3-methoxyphenyl-propanoate
   2.0% (w/w) (R)-mandelic acid 4-hydroxy-3-methoxyphenyl-propanoate
   Vehicle (both UV and Non-UV Control groups)
The treatment was repeated once daily for four consecutive days.
On day 6, two 4 mm punch biopsies were harvested and used as follows:
One was used for estimation of cell viability using the Resazurin Deep Blue Cell Viability Kit (cat. No. 424702) from BioLegend, USA. The biopsy was incubated for 4 hours in 900 μL fresh nutrient medium added 90 μL Resazurin Deep Blue, whereafter the medium was collected and transferred to a black fluorescence 96 well plate (Thermo Scientific 137101) and fluorescence was measured in a Spectramax ID5 at 550 nm excitation and 590 nm emission to establish cell viability.
The other biopsy was subjected to tissue homogenization on a GentleMACS™ with M tube from Miltenyi Biotec, Germany. This was followed by DNA extraction using the DNeasy® Blood & Tissue Kit (Qiagen, UK) following the standard protocol.
Double stranded DNA (dsDNA) was quantified using the Invitrogen™ Quant-iT™ dsDNA Assay Kit (cat. No Q33130) from Thermo Fisher Scientific, Denmark.
The amount of cyclobutane pyrimidine dimers (CPDs) in each biopsy was quantitated with specific antibodies employing the CPD ELISA kit from Cell Biolabs, USA. The assay was based on a standardized solution of dsDNA (4 μg/mL) from each sample.

### 2. UV-Induced DNA Damage in Human Reconstructed “Full Thickness” Skin.

In this model, a highly standardized 3D reconstructed human skin tissue (EpiDerm FT) based on differentiated primary human keratinocytes and fibroblasts was provided by Mattek Europe, Slovakia. These tissues were grown on a porous membrane and were cultivated in a nutrient medium in 6-well plates in an incubator at 37° C. and 5% CO₂.
Significant DNA damage was induced by placing the tissues in a UV-solar simulator (SOL 500 with UV-filter H2) from Dr. Hönle GmbH, Germany. This provided a combination of UVA and UVB comparable to natural sunlight.
4 groups of 4-5 explants were exposed to 100 mJ/cm² daily for two consecutive days. A control group of 3 explants without UV exposure was included to estimate the increase in DNA damage caused by the UV treatment.
Immediately after the last UV-treatment, the groups of explants were treated topically on the surface of the skin with 10 μL/explant of one of the following:
   3.0% (w/w) (R)-mandelic acid 4-hydroxy-3-methoxyphenyl-propanoate
   1.0% (w/w) (R)-mandelic acid 4-hydroxy-3-methoxyphenyl-propanoate
   0.5% (w/w) (R)-mandelic acid 4-hydroxy-3-methoxyphenyl-propanoate
   Vehicle
The treatment was repeated once daily for four consecutive days.
On day 6, two 4 mm punch biopsies were harvested used as follows:
One was used for estimation of cell viability using the Resazurin Deep Blue Cell Viability Kit (cat. No. 424702) from BioLegend, USA. The biopsy was incubated for 4 hours in 900 μL fresh nutrient medium added 90 μL Resazurin Deep Blue, whereafter the medium was collected and transferred to a black fluorescence 96 well plate (Thermo Scientific 137101) and fluorescence was measured in a Spectramax ID5 at 550 nm excitation and 590 nm emission to establish cell viability.
The other biopsy was subjected to tissue homogenization on a GentleMACS™ with M tube from Miltenyi Biotec, Germany. This was followed by DNA extraction using the DNeasy® Blood & Tissue Kit (Qiagen, UK) following the standard protocol.
Double stranded DNA (dsDNA) was quantified using the Invitrogen™ Quant-iT™ dsDNA Assay Kit (cat. No Q33130) from Thermo Fisher Scientific, Denmark.
The amount of cyclobutane pyrimidine dimers (CPDs) in each biopsy was quantitated with specific antibodies employing the CPD ELISA kit from Cell Biolabs, USA. The assay was based on a standardized solution of dsDNA (4 μg/mL) from each sample.

### Results:

UV-Induced DNA Damage in Human Skin Explants from a Middle-Aged Donor

The levels of CPDs in the three groups are shown in the table below:

| | Mean CPD | Reduction vs | |
|---|---|---|---|
| | Level | UV Control | Statistical |
| Group | (ng/ml) | (%) | Significance* |
| No UV Control | 2.3 | | |
| UV Control | 60.1 | | |
| 4.0% (R)-mandelic acid 4- | 16.1 | 73.2 | P < 0.0001 |
| hydroxy-3-methoxyphenyl- | | | |
| propanoate | | | |
| 2.0% (R)-mandelic acid 4- | 20.0 | 66.7 | P = 0.0001 |
| hydroxy-3-methoxyphenyl- | | | |
| propanoate | | | |

*Two-way ANOVA and Dunnett's test versus UV control
Both concentrations of topical (R)-mandelic acid 4-hydroxy-3-methoxyphenyl-propanoate displayed highly biologically and statistically significant reduction of CPDs indicating a significant enhancement of DNA repair.

### UV-Induced DNA Damage in Human Reconstructed “Full Thickness” Skin.

The levels of CPDs in the three groups are shown in the table below:

| | Mean CPD | Reduction vs | |
|---|---|---|---|
| | Level | UV Control | Statistical |
| Group | (ng/ml) | (%) | Significance* |
| No UV Control | 1.9 | | |
| UV Control | 85.3 | | |
| 3.0% (R)-mandelic acid 4- | 27.0 | 68.3 | P = 0.0014 |
| hydroxy-3-methoxyphenyl- | | | |
| propanoate | | | |
| 1.0% (R)-mandelic acid 4- | 32.8 | 61.5 | P = 0.0005 |
| hydroxy-3-methoxyphenyl- | | | |
| propanoate | | | |
| 0.5% (R)-mandelic acid 4- | 38.2 | 55.2 | P = 0.0001 |
| hydroxy-3-methoxyphenyl- | | | |
| propanoate | | | |

*Two-way ANOVA and Dunnett's test versus UV control
All concentrations of topical (R)-mandelic acid 4-hydroxy-3-methoxyphenyl-propanoate displayed highly biologically and statistically significant reduction of CPDs indicating a significant enhancement of DNA repair.

### Conclusion:

The data indicate that topically administered (R)-mandelic acid 4-hydroxy-3-methoxyphenyl-propanoate effectively penetrates the skin barrier and exerts a substantial enhancement of DNA repair. Besides relevance to skin aging, this effect indicates a strong potential for the treatment of skin cancer and other conditions associated with DNA damage.

### LIST OF REFERENCES

Aggarwal, P., Knabel, P., & Fleischer, A. B., Jr (2021). United States burden of melanoma and non-melanoma skin cancer from 1990 to 2019. Journal of the American Academy of Dermatology, 85(2), 388-395. https://doi.org/10.1016/j.jaad.2021.03.109
Arseni, L., Lombardi, A., & Orioli, D. (2018). From Structure to Phenotype: Impact of Collagen Alterations on Human Health. International journal of molecular sciences, 19(5), 1407. https://doi.org/10.3390/ijms19051407
Cakir, B. Ö., Adamson, P., & Cingi, C. (2012). Epidemiology and economic burden of nonmelanoma skin cancer. Facial plastic surgery clinics of North America, 20(4), 419-422. https://doi.org/10.1016/j.fsc.2012.07.004
Carlomosti, F., D'Agostino, M., Beji, S., Torcinaro, A., Rizzi, R., Zaccagnini, G., Maimone, B., Di Stefano, V., De Santa, F., Cordisco, S., Antonini, A., Ciarapica, R., Dellambra, E., Martelli, F., Avitabile, D., Capogrossi, M. C., & Magenta, A. (2017). Oxidative Stress-Induced miR-200c Disrupts the Regulatory Loop Among SIRT1, FOXO1, and eNOS. Antioxidants & redox signaling, 22(6), 323-344. https://doi.org/10.1039/ars.2016.6643
D'Arcangelo, D., Tinaburri, L., & Dellambra, E. (2017). The Role of p16^{INK4a} Pathway in Human Epidermal Stem Cell Self-Renewal, Aging and Cancer. International journal of molecular sciences, 16(7), 1591. https://doi.org/10.3390/ijms18071591
de Cabo, R., Liu, L., Ali, A., Price, N., Zhang, J., Wang, M., Lakatta, E., & Irusta, P. M. (2015). Serum from calorie-restricted animals delays senescence and extends the lifespan of normal human fibroblasts in vitro. Aging, 43), 152-166. https://doi.org/10.18632/aging.100719
Ezure, T., Sugahara, M., & Amano, S. (2019). Senescent dermal fibroblasts negatively influence fibroblast extracellular matrix-related gene expression partly via secretion of complement factor D. BioFactors (Oxford, England), 45(4), 556-562. https://doi.org/10.1002/biof 0.1512
Garcia-Peterson, L. M., Wilking-Busch, M. 3., Ndiaye, M. A., Philippe, C., Setaluri, V., & Ahmad, N. (2017). Sirtuins in Skin and Skin Cancers. Skin pharmacology and physiology 3(4), 216-224. https://doi.org/10.1159/000477417
Hernandez-Segura, A., Brandenburg, S., & Demaria, M. (2018). Induction and Validation of Cellular Senescence in Primary Human Cells. Journal of visualized experiments: JoVE, (136), 57732. https://doi.ora/10.3791/57732
Hornebeck W. (2003). Down-regulation of tissue inhibitor of matrix metalloprotease-1 (TIMP-1) in aged human skin contributes to matrix degradation and impaired cell growth and survival. Pathologie-biologie, 51(10), 569-573. https://doi.org/10.1016/j.patbio.2003.09.003
Idda, M. L., McClusky, W. G., Lodde, V., Munk, R., Abdelmohsen, K., Rossi, M., & Gorospe, M. (2020). Survey of senescent cell markers with age in human tissues. Aging, 12(5), 4052-4066. https://doi.org/10.18632/aging.102903
Imokawa, C., Nakajima, H., & Ishida, K. (2015). Biological mechanisms underlying the ultraviolet radiation-induced formation of skin wrinkling and sagging II: over-expression of neprilysin plays an essential role. International journal of molecular sciences, 164), 7776-7795. https://doi.org/10.3390/ijms16047776
Lee, Y. I., Choi, S., Roh, W. S., Lee, J. H., & Kim, T. G. (2021). Cellular Senescence and Inflammaging in the Skin Microenvironment. International journal of molecular sciences, 22(8), 3849. https://doi.org/10.3390/ijms22083849
Murphree R. W. (2017). Impairments in Skin Integrity. The Nursing clinics of North America, 52(3), 405-417. https://doi.org/10.1016/j.cnur.2017.04.008
Pfaffl M. W. (2001). A new mathematical model for relative quantification in real-time RT-PCR. Nucleic acids research, 2(9), e45. https://doi.org/10.1093/nar/29.9.e45
Pfaffl (2007) Relative quantification Real-time PCR, 3, 63-81 Published by International University Line (Editor: T. Dorak) https://www.gene-quantification.de/dorak-book-real-time-pcr-2006.pdf
Orioli, D., & Dellambra, E. (2018). Epigenetic Regulation of Skin Cells in Natural Aging and Premature Aging Diseases. Cells, 7(12), 268. https://doi.org/10.3390/cells7120268
Schmid-Wendtner, M. H., & Korting, H. C. (2006). The pH of the skin surface and its impact on the barrier function. Skin pharmacology and physiology, 19(6), 296-302. https://doi.org/10.1159/000094670
Sen, C. L., Reuter, J. A., Webster, D. E., Zhu, L., & Khavari, P. A. (2010). DNMT1 maintains progenitor function in self-renewing somatic tissue. Nature, 463(7280), 563-567. https://doi.org/10.1038/nature08683
Sriram, C., Bigliardi, P. L., & Bigliardi-Qi, M. (2015). Fibroblast heterogeneity and its implications for engineering organotypic skin models in vitro. European journal of cell biology, 94(11), 483-512. https://doi.org/10.1016/j.ejcb.2015.08.001
Tigges, J., Krutmann, J., Fritsche, E., Haendeler, J., Schaal, H., Fischer, J. W., Kalfalah, F., Reinke, H., Reifenberger, G., StOhler, K., Ventura, N., Gundermann, S., Boukamp, P., & Boege, F. (2014). The hallmarks of fibroblast ageing. Mechanisms of ageing and development, 138, 26-44. https://doi.org/10.1016/j.mad.2014.03.004
Waller, J. M., & Maibach, H. 1. (2006). Age and skin structure and function, a quantitative approach (II): protein, glycosaminoglycan, water, and lipid content and structure. Skin research and technology: official journal of International Society for Bioengineering and the Skin (ISBS) [and] International Society for Digital Imaging of Skin (ISDIS) [and] International Society for Skin Imaging (ISSI), 12(3), 145-154. https://doi.org/10.1111/j.0909-752X.2006.00146.x
Wang, A. S., Ong, P. F., Chojnowski, A., Clavel, C., & Dreesen, O. (2017). Loss of lamin B1 is a biomarker to quantify cellular senescence in photoaged skin. Scientific reports, 7(1), 15678, https://doi.org/10.1038/s41598-017-15901-9

1-13. (canceled)

14. A compound of the general Formula (I) or a pharmaceutical acceptable salt thereof 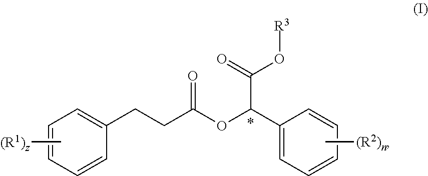
wherein
* denotes the (S) or (R) enantiomer or any mixture thereof;
z is an integer of 0, 1, 2, 3, 4 or 5 and R¹ is/are independently selected from F, OH, and C₁-C₄ alkoxy;
n is an integer of 0, 1, 2, 3, 4 or 5 and R² is/are independently selected from F, OH, and C₁-C₄ alkoxy;
R³ is selected from H and C₁-C₈ alkyl.

15. The compound according to claim 14, wherein z is an integer of 0-4.

16. The compound according to claim 14, wherein z is an integer of 0-3.

17. The compound according to claim 14, wherein z is an integer of 1-3.

18. The compound according to claim 14, wherein n is an integer of 0-4.

19. The compound according to claim 14, wherein n is an integer of 0-3.

20. The compound according to claim 14, wherein n is an integer of 0-2.

21. The compound according to claim 14, wherein n is an integer of 0-3 and z is an integer of 0-3.

22. The compound according to claim 14, wherein R¹ is/are independently selected from OH, and C₁-C₃ alkoxy.

23. The compound according to claim 14, wherein R² is/are independently selected from OH, and C₁-C₃ alkoxy.

24. The compound according to claim 14, wherein z is an integer of 0-3 and wherein the one or more R groups, when present, is/are located in position 3, 4 and/or 5 as shown in Formula (I)

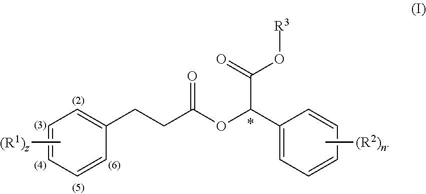

25. The compound according to claim 14, wherein n is an integer of 0-3 and wherein the one or more R² groups, when present, is/are located in position 3, 4 and/or 5 as shown in Formula (I)

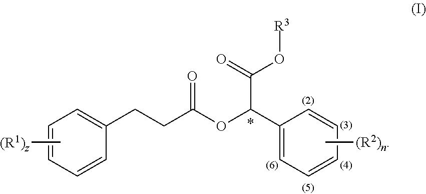

26. The compound according to claim 14, wherein R³ is selected as H or C₁-C₅ alkyl.

27. The compound according to claim 14, wherein R³ is selected as H or C₁-C₃ alkyl.

28. The compound according to claim 14, wherein R³ is H.

29. A method for the treatment of a dermatological disease, a rheumatological disease, a skin cancer, and/or a wound comprising administering a therapeutic effective amount of a compound according to claim 14.
